(19) United States
(12) Patent Application Publication
(10) Pub. No.: US 2008/0276327 A1
(43) Pub. Date: Nov. 6, 2008

(54) Methods and Compositions Related to Delivery of Chemical Compounds to Invertebrate Embryos

(75) Inventor: Aloisia T. Schmid, Salt Lake City, UT (US)

Correspondence Address: Ballard Spahr Andrews & Ingersoll, LLP, SUITE 1000, 999 PEACHTREE STREET, ATLANTA, GA, 30309-3915, US

(73) Assignee: UNIVERSITY OF UTAH RESEARCH FOUNDATION, Salt Lake City, UT (US)

(21) Appl. No.: 11/597,085

(22) PCT Filed: May 23, 2005
(86) PCT No.: PCT/US05/17900

§ 371 Date: Jun. 6, 2008

Related U.S. Application Data
(60) Provisional application No. 60/573,194, filed on May 21, 2004.

(51) Int. Cl. A01K 67/027

(52) U.S. Cl. 800/3; 800/21

(57) ABSTRACT

Disclosed are methods and compositions related to the delivery of chemical compounds to invertebrate embryos.

### CROSS-REFERENCE TO RELATED APPLICATIONS

This application claims priority to U.S. provisional application Ser. No. 60/573,194 filed on May 21, 2004. The 60/573,194 provisional patent application is herein incorporated by this reference in its entirety.

### I. BACKGROUND

In the pharmaceutical industry, chemical compound screening has been performed using cell-based assays in high throughput screening approaches. Template compounds are identified, optimized via medicinal chemistry and then introduced to whole animal systems for evaluation of toxicity. Judging the “success” of the new paradigm of drug discovery on the basis of published data has been difficult (Drews, 2000). High throughput screening has resulted in a large number of “hits,” However, very few leads and development compounds, if any, can be credited to the new drug discovery paradigm. (Jurgens, 1999). The major reason these hits have not proved useful for lead development is that the lead that is identified in the cell-based drug screening approach is very often later proved toxic in whole animals (Drews, 2000). Therefore, what is needed in the art are compositions and methods for delivering chemical compounds to invertebrate embryos, with the ability to do so in a high throughput screening assay.

### II. SUMMARY

Disclosed are methods and compositions related to the genetic modification of invertebrate embryos, as well as the delivery of chemical compounds to invertebrate embryos.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effects of gamma secretase inhibitors on Drosophila development. In all cases, anterior is to the left. All panels are confocal projections of whole mount embryos stained with rhodaminated antibodies directed against HRP, recognizing an antigen on the surface of all insect neurons. Panel A shows a horizontal view of wild type embryo demonstrating an intact central nervous system (CNS) with well organized peripheral nervous system, consisting of motoneuronal projections and sensory inputs. Panel B shows a diagram of the structures seen in wild type embryos. Panel C shows embryos exposed to 10 μM concentrations of gamma secretase inhibitors generate severe developmental defects, and never complete gastrulation. Panel D shows at 1 μM concentrations, the same drugs allow early cell populations to form but stall development prior to neurogenesis. Panel E shows 100 nM concentrations of gamma secretase inhibitors generate classic Notch neurogenic phenotypes. Panel F shows 10 nM concentrations generate slightly less severe neurogenic defects. Panels I (10 fM) and J (1 fM) show femtomolar concentrations of gamma secretase inhibitors generate defects only in peripheral nervous system pathfinding, a finding consistent with mildest hypomorphic alleles. Panels G and H show another gamma secretase inhibitor generated more severe defects in ventral nerve cord condensation.

FIG. 2 shows allelic series of the phenotypes associated with the misexpression of human APP fragments in the developing cells of the embryonic central nervous system. Pan-neuronal drivers were used to misexpress the amyloidgenic peptides associated with AD. The most severe phenotypes were consistently observed using ₛₛAbeta₁₋₄₂.

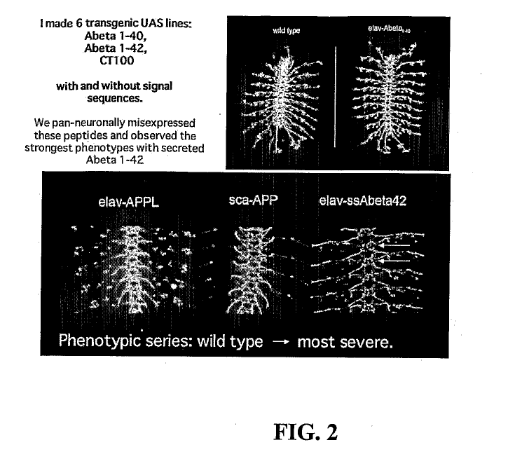

FIG. 3 shows that when DiI labeling experiments were performed to examine the single cell defects associated with the misexpression of Abeta peptides pan-neuronally, it was observed that the population of neurons most strongly affected were longitudinal projection neurons. These neurons consistently failed to extend beyond segment boundaries in this gain of function background. When staining was done with fascicular antibodies, clear breaks could be visualized at segment boundaries that typically extended for 25% of the Anteroposterior length of the hemisegment.

FIG. 4 shows a summary of an example of an Alzheimer's model (Example 2). This is an example of a phenotype on which a compound screen can be based.

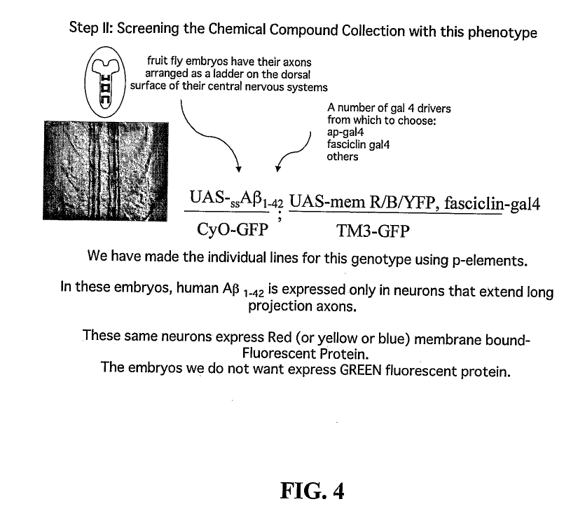

FIG. 5 shows a laser-based embryo sorter. The sorter uses a laser-based technology to detect the presence of GFP-tagged balancer chromosomes, eliminates those embryos from further study, but aliquots the embryos without such balancer chromosomes into each well of a 96 well plate, robotically. Each 96 well plate can be filled with 10 embryos per well, within 15 minutes.

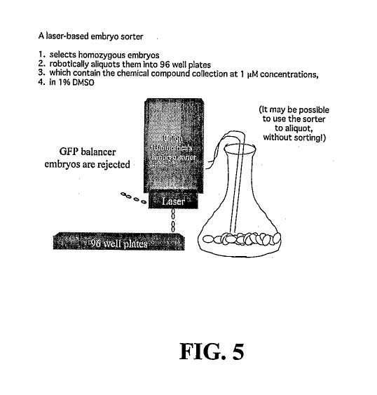

FIG. 6 shows a plate reader. Embryos develop in the drug, absorb the drug, and are then scored for the ability to extend axons across segment boundaries, as revealed by Red/Blue/Yellow membrane-tagged Fluorescent Protein expression patterns. An entire 96 well plate can be scanned and photographed in 10 minutes.

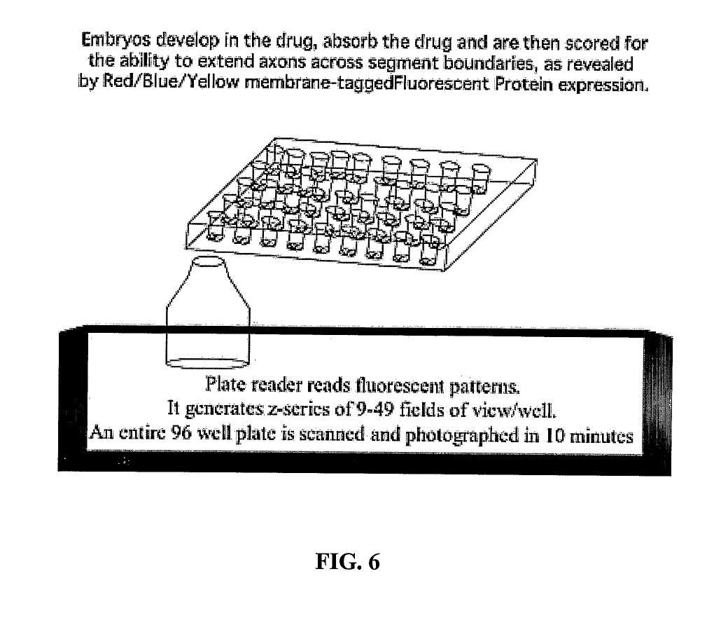

FIG. 7 shows a seed fill algorithm. Each image is divided into approximately 250,000 pixels, and each pixel is assigned a digital value for intensity, on a scale between 1-256. The computer evaluates the intensity of each pixel and then compares pairs of pixels and looks for neighbors that are equally bright. Fluorescent proteins never vary in their intensity, and plate readers can be used with seed fill algorithms. Essentially, the algorithm evaluates contiguous lines of pixels. So if membrane tagged fluorescent axons (pixels) are only 100 pixels long in the untreated controls, then a drug that ameliorates the phenotype can be expected to improve this number by what would be a statistically significant amount.

FIG. 8 shows RNAi for SMN generates motorneuronal and muscular defects in embryogenesis. Wild type stage 16 embryos of Drosophila melanogaster were filleted and stained with antibodies directed against a strongly conserved epitope of human SMN1; these were counterstained with A488-conjugated phalloidin (panels A-C) and photographed on a Biorad Radiance 2000 confocal microscope. The SMN1 antibodies appear to recognize an epitope in Drosophila that is specific to neurons, their axons (asterisk, panel B) and muscles (arrow, panel B). RNAi experiments at 5 uM concentrations (panels D-F) revealed that the antibody staining is specific, as its cellular specificity disappears and is replaced by relatively low level non-specific background staining (panel E). Motoneuronal projections, visible as regular processes exiting from the CNS (panel A) are absent in RNAi backgrounds (Compare panels A and D). Furthermore, muscles continue to extend myopodia in RNAi backgrounds (arrow, Panel D), apparently because their extension has not been suppressed by successful motoneuronal innervation, which would normally be intact by stage 16. At 10 uM concentrations (panels G-I), little CNS cytoarchitecture remains and SMN staining is non-specific and confined to overlying fat cells. The number of segments in which recognizeable muscles are formed is less than 40%; these panels demonstrate that muscles can form, but continue to exhibit abnormal morphology, including the prolonged extension of myopodia. In all cases, anterior is down. Panels A-C and G-I are photographed at 60×. Panels D-F were photographed at 84× (indicated by size bars). Single cell labelings are in progress to determine whether or not motorneurons do indeed fail to form, or extend axons.

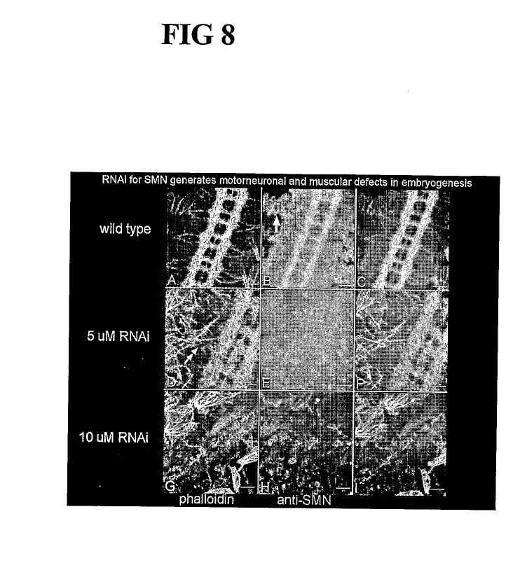

FIG. 9 shows that at the single cell level, motoraxons fail to exit the CNS. On the left are DiI lineages that were created in backgrounds in which gene function had been removed by RNAi. (See figure X for method) The embryonic CNS contains 34 stem cells (neuroblasts, NB) that generate invariant lineages. NB 1-2 in thoracic segments generates the DC motorneurons which are the most robust motomeurons of the CNS and which innervate the mouth hook muscles. Larvae are able to retract their heads to the insides of their bodies as a defensive mechanism, when touched or threatened, by a relfelx mediated by the DC motorneuronal innervation mouth hook muscles. In the figure provided, the wild type lineages (including thoracic lineages with robust DC motomeurons) are depicted in the wild type panels on the right, whereas RNAi mutant clones are presented on the left. Even these very large calibre motomeurons are not able to exit the CNS when SMN function is removed. All of the lineages of the ventral nerve cord were examined, and the same failure to extend axons to target fields were found in every lineage examined at high doses. A dose responsiveness to these phenotypes was also found; lower doses of dsRNA allowed some motomeurons to escape this axonal failure. At 5 uM concentrations, virtually all motorneurons were affected identically, as shown in this figure.

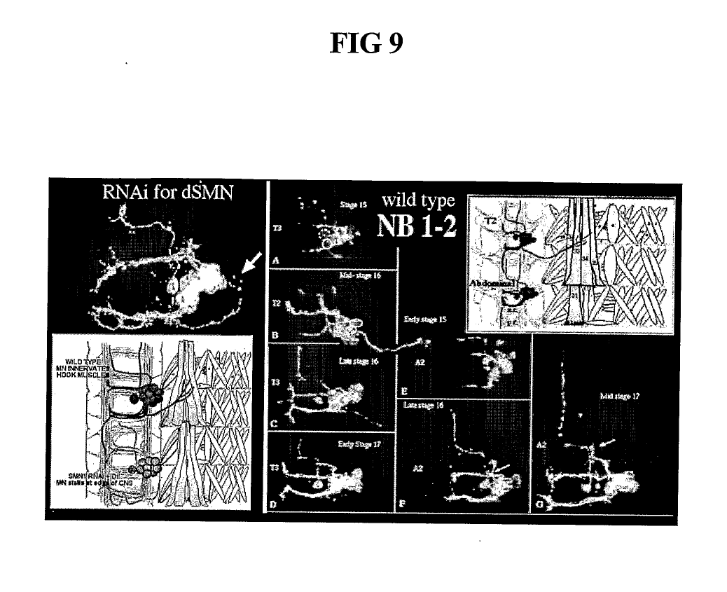

FIG. 10 shows RNAi followed by lineage tracing. Embryos were injected with double-stranded RNA at presyncytial blastoderm stages. They were then allowed to develop to stage 8 at 16 degrees C. Then they were injected with 1 micron droplets of DiI suspended in vegetable oil. Single neurectodermal cells take up this dye and are able to transfer this dye to other cells only by lineal transfer during cytokinesis. In this way the entire lineage was visualized, in loss of function backgrounds. The RNAi can be used to inactive one or more genes simultaneously.

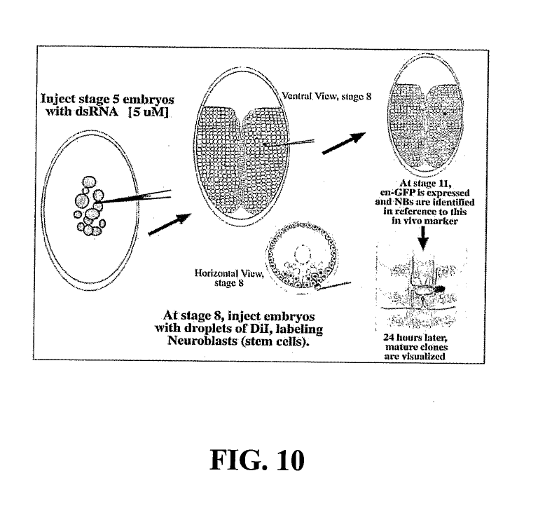

FIG. 11 shows filleted Drosophila embryos were fixed and stained with Monoclonal antibody 22C10, staining a known subset of neurons in the embryonic CNS. Panel A: wild type, Panel B: RNAi for Flywolf, C: Silver, a Drosophila ortholog of Carboxypeptidase E, and D: GABA B1, the Drosophila ortholog of the AGAB receptor. Carboxypeptidase E and GABA B1 are putative interactors with Wolfram. Drosophila alleles were tested for similarity of phenotype, and even though the Flywolf RNAi experiments demonstrate cell specific losses, defects observed with carboxypeptidase and GABA B1 orthologs did not generate phenotypes that appeared to place them in the Flywolf pathway. Thus, Flywolf reveals a new genetic pathway related to depression and hearing defects.

FIG. 12 shows embryos were injected with dsRNA. They were allowed to develop to stage 8 and were then injected with single droplets of DiI suspended in Wesson Vegetable oil. In wild type embryos, NB 2-4 generates a single contralaterally projecting motomeuron, and a small cluster of local interneurons. In Flywolf RNAi backgrounds, the motomeuron is relatively unperturbed, but the local interneurons fail to extend axons, both ipsilaterally and contralaterally. This phenoptye is conserved with those reported in human Wolfram Syndrome patients.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

The term “transgene” is used herein to describe genetic material which is artificially inserted into the genome of an invertebrate cell. The transgene encodes a product that, when expressed in embryos, gives rise to a specific phenotype. Generally, the transgene encodes a transcription factor or mimetic thereof having the desired result.

The terms “alleviating” or “ameliorating” denotes a lessening of an effect of a condition or disorder, such as a detrimental affect, in the animal or situation having the effect, such as the invertebrate embryo. This lessening of the effect can occur phenotypically or genotypically.

The term “therapeutically effective” means that the amount of a composition used is of sufficient quantity to ameliorate at least one effect, such as the cause, of a disease.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS AND COMPOSITIONS

Disclosed are methods and compositions for drug screening in insects, such as Diptera, such as Drosophila melangastor, and hymenoptera, such as the common honey bee. These methods and compositions allow for high-throughput screening of compounds for characteristics such as toxicity and effectiveness, and in certain embodiments, the screening in the insects can correlate with target disease states, in for example, humans, which is aided by correlative data. Disclosed herein, are methods and compositions that are associated with human neurodegenerative diseases, wherein models in the fly are predictive of phenotypes of these diseases. Furthermore, these predictive states in certain embodiments, are identifiable at the embryo stage of the insect. Diseases such as Parkinson's and Alzheimer's are disclosed. These methods and compositions are related to the understanding disclosed herein that embryos of insects, such as those in Diptera and Hymenoptera, can be treated in certain ways, to allow for the entry of molecules into the embryo, without destroying the embryo or destroying the development potential of the embryo. This allows for the screening methods disclosed herein. Furthermore, the insects, can be genetically manipulated, such as the Diptera and Hymenoptera, so that these screens can be performed on particular genetic backgrounds. For example, the insects can be genetically manipulated such that the insects harbor a human transgene, such that when the gene is expressed when a phenotype arises in the insect that is predictive of a particular disease state in humans, such as a neurodegenerative state, such as Parkinson's or Alzheimer's. In association with this, disclosed are particular transgenes that are shown herein to have a predictive phenotype in insects for the disclosed human disease states.

Since, in certain embodiments, high-throughput screening is desired, also disclosed herein are machines and algorithms to facilitate the sorting of insect embryos, the categorizing of insect embryos, and the analyzing of the embryos, for example.

In part, provided below is a discussion of general drug screening and high-throughput drug screening. Then a discussion of the developmental pathway of insects, such as Diptera, along with important developmental steps related to the embryo stage. Also provided is a discussion of the various parts of the disclosed compositions, such as the altered insect embryos, and the various permutations and alterations on the screening methods disclosed herein. In addition, there is a discussion of a number of the important aspects of, for example, molecular biology techniques and structural aspects of biological macromolecules, such as proteins. Molecules that can be used for screening are also discussed.

1. Drug Screening

Compound screening is typically performed using cell-based assays. Template compounds are identified, optimized via medicinal chemistry and then introduced to whole animal systems for evaluation of toxicity. Previously, drug discovery was performed in whole animal models, usually rodents. When whole animal studies became prohibitively expensive or too time consuming, rodent models were replaced with cell-based assays so that high throughput screening (HTS) approaches could be developed. High-throughput screening can be any screen that allows for the analysis of at least 2000, or more compounds in a day. In certain high-throughput screening methods, more than one compound, such as 10, 20, 30, 40, 50, 60, 70, 80, 90, 100, 150, 200, 250, 300, 350, 400, 450, 500, 600, 700, 800, 900, 1000, 2000, 3000, 4000, 5000, or more compounds are screened simultaneously, meaning analyzed in the same set of experimental manipulations.

2. Parkinson's Disease

Parkinson's Disease (PD) is the second most common neurodegenerative disease in aging populations. It affects dopaminergic neurons specifically and is characterized by the presence of Lewy bodies and Lewy neurites, the major component of which is α-synuclein. The disease is further characterized by severe behavioral deficits including dyskinesia and tremor. Hereditary forms of Parkinson's disease have been linked to mutations in proteasomal proteins (e.g., parkin, an E3 ubiquitin ligase) and to mutations in α-synuclein, such as the A53T α-synuclein mutation, and the A30P α-synuclein mutation.

3. Alzheimer's Disease

Alzheimer's Disease is even more common than Parkinson's Disease, affecting an estimated 35% of Americans over the age of 75, and 50% of Americans over the age of 80. It is correlated with the deposition of amyloid plaques containing cleaved products of the Amyloid Precursor Protein (APP); these cleavage products (Abeta₁₋₄₀ and Abeta₁₋₄₂) are the result of cleavage in APP's extracellular domain by beta-secretase (BACE) and in the transmembrane domain by gamma-secretase.

4. Flies as Models of Human Disease

The introduction of human amyloidgenic peptides into the fly have been observed to produce reliable models of human neurodegenerative diseases (Feany and Bender, 2000; Pendelton et al, 2002; Bonini 2001). The fly model of Parkinson's Disease relies on the expression of human mutant α-synuclein in the fly's own dopaminergic neurons. These Drosophila neurons, expressing human mutant α-synuclein, are observed to die in an age-related manner, whereas non-dopaminergic neurons are not affected. α-synuclein-positive fibrils are observed to form Lewy-body-like structures in the brains of these flies, something that has never been observed in any other animal model of Parkinson's Disease. Flies expressing human mutant α-synuclein also experience behavioral deficits that mimic those observed in PD patients, namely dyskinesia and tremor.

When treated with the same drugs used to treat human PD patients, flies respond with the same dose-responsiveness and in the same efficacy order observed in humans. In other words, flies respond to drugs in the same dose ranges used for humans (normalized for body weight), and respond well to drugs that are effective in humans, and show less amelioration of their phenotypes when exposed to drugs that are not particularly effective in human patients (Pendleton et al, 2002). When modifier screens were performed using this model, Hsp70 proteins were identified as being able to suppress (albeit incompletely) the age-related neurodegeneration of human mutant α-synuclein in dopaminergic neurons when simultaneously expressed in the same cells. When human PD brains were probed for the expression of these Hsp70 proteins, they were indeed identified as colocalizing with mutant α-synuclein in the Lewy bodies of these brains, revealing for the first time that the fly could be used to predict the pathology of the human (Auluck et al, 2002).

Thus, the Drosophila model of PD generates a convincing replica of human age-related dopaminergic neurodegeneration that responds to compounds in a way that suggests the underlying cell biology is identical in both systems; genes/proteins identified as modifying these pathologies in the fly have been identified in identical contexts in humans. However, drug studies using age-related neurodegeneration as a read-out are exceptionally slow, laborious and costly, limiting the number of compounds that can be screened in large adult flies (Reifegerste et al. WO 03/028446, 2003).

The value of Drosophila as a screening system for evaluating the biological activities of chemicals has been well-documented (Schulz, et al., 1955). Previously, small numbers of chemical substances were administered to larvae or flies by feeding, and the adult flies were then analyzed for survival and for phenotypic alteration. Alternatively, flies were injected with compounds and then the adult flies were monitored. These methods, however, did not permit high-throughput screening, nor permit the directed search for small molecular weight compounds that interfere with a specific morphogenetic pathway related to a human disease condition.

What is needed in the art is the ability to introduce chemical compounds into invertebrate embryos, which improves the accuracy and speed of both template identification and the evaluation of toxicity, as well as analysis of the effectiveness of compounds. In addition, these systems, if appropriately modified will allow for the identification of drugs which can treat a wide variety of diseases, such as neurodegenerative diseases. Such a system would allow for high throughput screening approaches in genetic model systems, using minute concentrations of compounds.

5. Drosophila Genetics

The use of invertebrate model organism genetics and related technologies can greatly facilitate the elucidation of biological pathways (Scangos, 1997). Drosophila melanogaster is the premier model system of genetics. It has a number of advantages that make it ideally suited to compound screening: 1) Drosophila are fast-growing, 2) Dropsophila generate large populations of offspring, and 3) Drosophila cost very little to maintain. Furthermore, because embryonic development requires only 24 hours at room temperature, the growth and maturity of new populations occurs very rapidly, and the phenotypic analysis can proceed. Drosophila have within them, and can accept, transposable elements allowing for the easy introduction of foreign DNA (including human) into its genome. Drosophila also have fluorescently tagged balancer chromosomes allowing for the easy maintenance of lab stocks, the detection of genotype in the embryo and the sorting of heterozygotes from homozygotes. The invariant central nervous system (CNS) lineages are known (Schmid et al, 1999), making single cell phenotypic analysis of CNS defects relatively easy. Furthermore, a developing embryo is an organism with complex organ systems, and almost the entire genome is expressed during embryonic development, making toxicity observed in an embryo reflective of toxicity in an adult, as disclosed herein.

The expression of human disease genes or their homologs within developing Drosophila larva models as shown herein can have distinct effects on Drosophila. The effects of these genes in human cells can be elucidated or mimicked in Drosophila, and subsequently the phenotypes which are modified by either the mutations within these interacting genes, or by compounds which block the function of the corresponding gene product, can be produced. These gene products are prime candidates as targets for small compounds which interfere with their function.

6. Screening Assays

Disclosed herein are high throughput assays with a readout system comprised of invertebrate embryos which are genetically-sensitized for a specific disease pathway, such as a human disease pathway.

Disclosed herein are methods for conducting a wide variety of biological assays using invertebrate embryos, such as insects, such as Endopterygota, such as Diptera, such as Drosophila melangastor, or Hymenoptera, such as bees. These biological assays can be conducted in a number of ways, including, but not limited to, high throughput screens or screens containing one, two, or more embryos. Large numbers of compositions can be screened simultaneously, or compositions can be screened one at a time (individually). Biological assays can also be used to detect interaction between compositions, as well as to analyze the function of molecules, such as small molecular weight compounds. Large numbers of compounds can be screened for biological/therapeutic activity in a rapid, quantitative and highly efficacious manner. Such assays can be conducted on chemical compounds or any molecule of biological interest, included but not limited to drug candidates, such as those found in combinatorial libraries, siRNAs, and antibodies. High-throughput screening of collections of chemically-synthesized molecules and of natural products can be carried out using the disclosed methods. Furthermore, these methods can be performed on embryos that have been specially engineered to display a desired phenotype. For example, the phenotype can be a phenotype that correlates with a known human or other non-fly animal condition, which has an orthologous condition or response which is correlative within the Drosophila. In this fashion, models for human diseases can be made, within the drosophila, and specifically, a phenotype that is identifiable within the embryo of the Drosophila can be utilized. Such phenotypes are disclosed herein.

Also provided are methods for profiling multiple biological responses of drug candidates on invertebrate embryos. The potent and specific biological activities of many low molecular weight molecules make these molecules attractive starting points for therapeutic drug discovery (Hirschmann, et al., 1991). Also disclosed are methods of identifying pharmacological agents for the treatment of disease. For example, by working in the disclosed systems, toxicology and efficacy testing can be performed simultaneously, eliminating the need for doing, for example preclinical data, to arrive at a candidate therapeutic compound, only to have that compound be untractable in a therapeutic setting because of a later identified toxicity problem associated with the compound.

In certain embodiments, the methods involve administering a compound to an insect embryo. Typically, the insect embryo has been manipulated. For example, in certain embodiments, the insect embryo first has a hard shell, external to the vitelline membrane, which is removed or degraded using any means. Then, for example, the vitelline membrane is compromised, for example, with any material, such as DMSO, so that materials can enter into the embryo. Thus, disclosed are methods that make and/or utilize embryos that have been treated in this way.

a) Development and Embryos

Any invertebrate animal embryo can be used with the methods disclosed herein. Invertebrate organisms include, but are not limited to, arthropods; particularly insects species such as Drosophila (such as the fruit fly D. melanogaster), Apis (such as the honeybee A. mellifera), and Leptotarsa; acarids; crustacean; mollusks; worms such as Caenorhabditis elegans; coelomates and pseudocoelomates. Of particular use is the insect model organism, Drosophila melanogaster.

Drosophila development takes approximately ten days, from the fertilization of an egg by a sperm cell to the eclosion, or hatching, of a mature adult. There are four major stages of development in that ten day period of time, and each of these is separated by a clear, punctuated event.

Embryonic development occurs within the first 24 hours following fertilization, and encompasses 17 stages, as seen in Table 1 (http://www.flybase.org; http://sdb.bio.purdue.edu/fly/aimain/1aahome.htm). Embryonic development ends with hatching from the egg shell, with a fully functioning larvae. The hatching event is a test of development, as most genetic defects render an embryo incapable of moving on to larval development.

There are two major developmental strategies that have evolved through time: hemimetabolous (partial metamorphic changes) and holometabolous. Hemimetabolous reproduction is the type in which post-hatching developmental stages are primarily enlargement stages and do not require major structural rearrangement or maturation. Fruit flies are holometabolous organisms, meaning embryonic development occurs over a much shorter timespan. The eggs are smaller, invested with far fewer calories, requiring the developing embryo to hatch earlier, but also demanding much less of it in terms of its behavioral repertoire. Fly larvae find and store calories from external sources, as these calories have not been maternally provided.

There are three larval stages in Drosophila. These are referred to as larval instars. Each transition in larval development requires a molting, a shedding of the exoskeleton and the formation of a new larger skin (exoskeleton). Each instar is therefore defined from one molting to the next. The first instar spans the period from hatching from the egg, to its first instar molting, and this requires approximately 24-26 hours at room temperature. The second instar goes from the end of the first instar to the start of the third instar, at the second molting and this requires approximately 36 hours. The final instar stages require about 48 hours and is the major feeding stage of Drosophila development, the larvae having grown from approximately 0.5 mm in length to 4.5 mm in length, and increasing in body mass approximately 50-fold and with approximately 80% of the growth occurring during third instar larval stage.

White pupae are the earliest stages of pupae development. During larval and pupal development, the tissues fated for perdurance in the adult become polytene, expressing thousands of copies of each gene, in order to facilitate rapid transcription and growth. The only tissues which will endure to adulthood that do not become polytene are the cells of the central nervous system. The larval polytene tissues are referred to as imaginal discs and histoblast nests. During pupal development, the tissues of the larvae that are not CNS, imaginal discs or histoblast nests, liquefy, and are then remodeled into new configurations. All of pupal development requires 4-5 days. The first 65% of pupal development is white pupal stages, in which these remodeling events occur, and the final pupal stages are referred to as pigmented pupal stages, and are melanization stages, where cuticle is formed and pigmented, where final eye development and photoreceptor maturation occurs (as visualized by eye pigment deposition) and where wing, leg, and antennal structures are melanized. Eclosion is the final hatching from the pupal case (the chrysalis) and occurs almost exactly ten days from fertilization.

The present methods can be used with an embryo in any of the various stages of development described above. In one embodiment, the embryos are collected from a cage containing many flies, such as thousands or millions of flies. In one embodiment, these cages comprise plastic cylinders approximately one foot in diameter. One end is covered with a mesh circle, allowing free air circulation and at the other end are mesh sleeves allowing easy access to the interior of the cage. The sleeves allow collection plates to be inserted and easily retrieved. Large petri dishes with yeast as a food source are inserted, allowed to remain for approximately 30 minutes and are then removed. Optimally functioning cages can produce plates with millions of embryos within in a 30 minute time span. An automated embryo collector (such as these cages described above) can be used. Embryos can be collected at various times. The embryos can be collected, for example, every 5, 10, 15, 20, 30, 40, 50 or 60 minutes. Embryos can be collected within 30-60 minutes of egg laying so as to allow drugs to penetrate embryos from the earliest stages.

The chorion, or the “shell” on the embryos, can be removed after the embryo is collected. The chorion is a soft waxy shell external to the vitelline membrane of the egg. The chorion contains a number of subparts, such as the endochorion, exochorion, and inner chorionic layer. The various layers of the chorion can be used as assays for follicular cell defects, the follicle cells being the ones that secrete the chorion. Drosophila embryos are referred to as having a chorionic membrane, which is completely removed in 100% bleach. When dechorionating an embryo, one is degrading the chorion layer. There are many known methods of dechorionation, including exposure to bleach or sticky tape. When exposed to bleach, the embryo can, for example, be soaked in a 10%, 20%, 30%, 40%, 50%, 60%, 70%, 80%, 90%, or 100% solution. The embryos can be soaked for 1, 2, 3, 4, 5, 6, 7, 8, 9, 10, 15, 30, 45, or 60 or more minutes. Alternatively, the embryos can be dechorionated by brushing the embryos onto a piece of tape, such as double-stick tape, and then removing them, leaving the chorion behind. Tungsten needles can also be used. After dechorionation, the embryo can be treated to facilitate the uptake of a composition. For example, the embryo can be subjected to electroporation, or exposed to a substance that increases the permeability of the embryo, such as an alkaline solution such as soap, tritonX-100 or Tween20, or, preferably, DMSO.

In one embodiment, the embryo can be exposed to the alkaline solution, such as DMSO, at a concentration of less than or equal to 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, or 15% or greater. Preferably, the embryo is exposed to 1.0%-5.0% DMSO.

The embryos can be exposed to the alkaline solution, such as DMSO, at any temperature, for example 10° C., 12° C., 14° C., 16° C., 18° C., 20° C., 22° C., 24° C., 25° C., 26° C., 27° C., 28° C., 30° C., 32° C., 34° C., or 36° C. The warmer the temperature the greater the effect of the DMSO on the embryo. The length of time the embryos are exposed to DMSO can vary, as well. For example, the embryos can be exposed 12, 18, 24, 30, 36, 42, or 48 hours or more, and can be exposed before, during, or after exposure to the compositions of interest. Also, the longer the time the embryos are exposed the greater the effect of the DMSO. For example, a typical protocol can include exposing the embryo to DMSO for 36 hours at 18° C. Another protocol can include exposing the embryo to DMSO for 24 hours at 25° C.

The embryos can be, exposed from the earliest stages of embryonic development. This allows for maximum uptake and exposure to the compositions of interest. For example, if embryos are collected and sorted before embryonic stage 5, they are still presyncytial blastoderms. This allows them to have compositions for screening available to them at the points at which they segregate CNS tissues, for example. Table 1 shows the 17 classic stages of embryonic development

TABLE 1
Drosophila Embryo Stages of Development

| stage | minutes after | |
|---|---|---|
| number | fertilization | developmental activity |
| 1 | 0-15 | Pronuclear fusion |
| 2 | 15-70 | Preblastoderm (mitotic cycles 1-9) - early cell division - start of |
| | | cleavage |
| 3 | 70-90 | Pole bud formation - nuclear division 9 |
| 4 | 90-130 | Syncytial blastoderm (mitotic cycles 10-13) - end of cleavage |
| | | divisions |
| 5 | 130-180 | Cellularization of the blastoderm |
| 6 | 180-195 | Gastrulation to form mesoderm and endoderm - pole cells included in |
| | | posterior midgut primordium |
| 7 | 195-200 | Germ band elongation - lengthening of the ventral epidermis |
| 8 | 200-230 | Rapid germ band elongation - start of first postblastoderm mitosis - |
| | | ends with mesodermal parasegmentation |
| 9 | 230-260 | Slow germ band elongation - segmentation of neuroblasts - end of |
| | | first and start of second postblastoderm mitosis - cephalic furrow |
| | | formation |
| 10 | 260-320 | Gnathal and clypeolabral lobe formation (head features) - stomodeal |
| | | invagination - end of second and start of third postblastoderm mitosis |
| 11 | 320-440 | Epidermal parasegmentation evident - tracheal pits invaginate - |
| | | mesectodermal cell ingress - end of third postblastoderm mitosis - |
| | | end of neuroblast formation |
| 12 | 440-580 | Germ band retraction - optic lobe invagination - ventral closure - |
| | | segment formation - fusion of anterior and posterior midgut |
| 13 | 560-620 | End of germ band retraction - CNS and PNS differentation |
| 14 | 620-680 | Dorsal closure of midgut and epidermis - head involution begins |
| 15 | 680-800 | End of dorsal closure - head involution - discs invaginate - cuticle |
| | | deposition begins - dorsal epidermal segmentation |
| 16 | 800-900 | Advanced denticles visible - Shortening of the ventral nerve cord |
| 17 | Lasts until | The tracheal tree fills with air - Retraction of the ventral cord |
| | hatching | continues |
| Hatch | 21-22 hours | Hatch to first instar larva |

b) Genetic Manipulation of Insects and Insertion of Transgenes

In certain embodiments, it is desired that the insect, such as the Drosophila, have been genetically manipulated prior to being used in one of the screening methods. For example, often, the insect will have one or more transgenes inserted into its genome so that a particular genetic product can be produced. Typically genetic manipulation of insects involves constructs having three components, the transferring element, understood as the vector component, a promoter region, which can also include an enhancer, and the sequence to be expressed, such as a sequence which encodes for a protein, or for example, a siRNA.

The genetic modification may be produced by a naturally-occurring, non-wild-type allele of a specific gene which is isolated from a genetic mutagenesis screening assay well-known to those individuals skilled within the art. Alternatively, the genetic modification may be produced by genetic manipulation using genetic recombination/molecular biological techniques known to those skilled within the art. The preferred genetic modification is a non-wild-type allele which, when present in the heterozygous state, results in an altered phenotype that is dose dependent. As utilized herein, the term “dose dependent” is designated as meaning that the genetically-sensitized Drosophila strain exhibits an observably different phenotype for each specific genetic state when it possesses either none, one or two copies of the modified allele of the gene of interest. Loss of function phenotypes are also included. For example, in SMN (Spinal motorneuronal atrophy), the human gene exists as an almost perfect duplication, known as SMN1 and SMN2 (SEQ ID NOs:9-14). In patients homozygous for loss of SMN1, duplications of SMN2 can rescue the phenotype, but only partially. This syndrome can be duplicated using Drosophila versions of these genes. The SMN2 gene is part of a 500 kb inverted duplication on chromosome 5q13. This duplicated region contains at least four genes and repetitive elements which make it prone to rearrangements and deletions. The repetitiveness and complexity of the sequence have also caused difficulty in determining the organization of this genomic region. The telomeric and centromeric copies of this gene are nearly identical and encode the same protein. While mutations in the telomeric copy are associated with spinal muscular atrophy, mutations in this gene, the centromeric copy, do not lead to disease. This gene may be a modifier of disease caused by mutation in the telomeric copy. The critical sequence difference between the two genes is a single nucleotide in exon 7 which is thought to be an exon splice enhancer. It is thought that gene conversion events may involve the two genes, leading to varying copy numbers of each gene. The full length protein encoded by this gene localizes to both the cytoplasm and the nucleus. Within the nucleus, the protein localizes to subnuclear bodies called gems which are found near coiled bodies containing high concentrations of small ribonucleoproteins (snRNPs). This protein forms heteromeric complexes with proteins such as SIP1 and GEMIN4, and also interacts with several proteins known to be involved in the biogenesis of snRNPs, such as hnRNP U protein and the small nucleolar RNA binding protein. Four transcript variants are produced by this gene: centromeric Isoform Variants: Isoform a: Transcript Variant: This variant (a) lacks exon 7, which leads to a premature termination codon. This variant is thought to be the predominant transcript produced by this copy of the gene. Apparently, no Drosophila ortholog has been identified, but vertebrate SMN2 has been shown to be regulated by transformer (a drosophila sex determination gene) and can regulate the splicing of double sex (another gene in that sex regulation pathway). The theory is that SMN2 arose as a perfect duplication of SMN1 during one of the metameric duplications, but that a single base change resulted in an alternative splice site, resulting in these various isoforms. This would account for why Drosophila orthologs do not exist (there are two metameric duplications separating the invertebrates from vertebrates, so the formation of SMN2 and its complicated alternative splice sites would not yet have happened in the fly.) Other “loss of function” phenotypes are included as well.

7. Transferring Element

Transgenic flies can be prepared using any convenient protocol that provides for stable integration of the transgene into the fly genome in a manner sufficient to provide for the requisite spatial and temporal expression of the transgene, i.e. in embryonic neuroblasts. A number of different strategies can be employed to obtain the integration of the transgene with the requisite expression pattern. Generally, methods of producing the subject transgenic flies involve stable integration of the transgene into the fly genome. Stable integration is achieved by first introducing the transgene into a cell or cells of the fly, e.g. a fly embryo. The transgene is generally present on a suitable vector, such as a plasmid. Transgene introduction may be accomplished using any convenient protocol, where suitable protocols include: electroporation, microinjection, vesicle delivery, e.g. liposome delivery vehicles, and the like. Following introduction of the transgene into the cell(s), the transgene is stably integrated into the genome of the cell. Stable integration may be either site specific or random, but is generally random.

Where integration is random, the transgene is typically integrated with the use of transposase. In such embodiments, the transgene is introduced into the cell(s) within a vector that includes the requisite P element, terminal 31 base pair inverted repeats. Where the cell into which the transgene is to be integrated does not comprise an endogenous transposase, a vector encoding a transposase is also introduced into the cell, e.g. a helper plasmid comprising a transposase gene, such as pTURBO (Steller & Pirrotta, 1986). Methods of random integration of transgenes into the genome of a target Drosophila melanogaster cell(s) are disclosed in U.S. Pat. No. 4,670,388, the disclosure of which is herein incorporated by reference.

In those embodiments in which the transgene is stably integrated in a random fashion into the fly genome, means are also provided for selectively expressing the transgene at the appropriate time during development of the fly. In other words, means are provided for obtaining targeted expression of the transgene. To obtain the desired targeted expression of the randomly integrated transgene, integration of particular promoter upstream of the transgene, as a single unit in the P element vector may be employed. Alternatively, a transactivator that mediates expression of the transgene may be employed. Of particular interest is the GAL4 system as described herein.

As mentioned above, an example of a transposable element is the P element (Rubin and Spradling 1982, Spradling and Rubin 1982). The gene of interest is placed between P element ends, usually within a plasmid, and injected into pre-blastoderm embryos in the presence of transposase. This P element, with the gene as cargo, then transposes from the plasmid to a random chromosomal site. P-elements are small transposons with terminal 31-bp inverted repeats, and the element generates 8-bp direct repeats of target DNA sequences upon insertion. The complete element is 2907 bp and is autonomous because it encodes a functional transposase. Incomplete P elements have lost the transposition ability because the transposase has been mutated. But if a complete (autonomous) element exists in the same cell as an incomplete (non-autonomous) element, then the incomplete element can transpose because of the presence of the transposase in the cell (Ashburner 1989, Spradling 1986).

Examples of other transposable elements include piggyBAC and Mariner. The piggyBAC element is 2.4 kb in length and terminates in 13 bp perfect inverted repeats, with additional internal 19 bp inverted repeats located asymmetrically with respect to the ends (Cary et al. 1989). The initial sequence analysis of the piggyBAC element revealed a potential RNA polymerase II promoter sequence configuration, typical Kozak translational start signal, and two apparently overlapping long open reading frames. Mariner belongs to a superfamily of DNA-based transposons that includes the C. elegans Tc1 element. It is small (1.3 kb), encoding one protein (the transposase) flanked by 28 bp inverted repeats (Medhora, M. M., Maruyama, K. and Hartl, D. L. (1991) Genetics 128:311.)

(1) Promoter Element

In certain embodiments, the expression of the transgene is targeted to occur in a non-adult stage of the animal. Typically, the transgene is stably integrated into the genome of the animal in a manner such that its expression is directed both spatially and temporally to the desired cell type and the correct developmental stage, i.e. to expression in embryonic neuroblasts, but the transgenes can also be expressed constitutively as disclosed herein. Specifically, the subject transgene is stably integrated into the genome of the animal under the control of a promoter that provides for expression. The transgene may be under the control of any convenient promoter that provides for this requisite spatial and temporal expression pattern, where the promoter can be endogenous or exogenous. For example, a suitable promoter is the promoter located in the Drosophila melanogaster genome at position 86E1-3.

Another suitable promoter of the Drosophila origin includes the Drosophila metallothionein promoter (Lastowski-Perry, 1985). This inducible promoter directs high-level transcription of the gene in the presence of metals, e.g., CuSO₄. Use of the Drosophila metallothionein promoter results in the expression system of the invention retaining full regulation even at very high copy number. This is in direct contrast to the use of the mammalian metallothionein promoter in mammalian cells in which the regulatory effect of the metal is diminished as copy number increases. In the Drosophila expression system, this retained inducibility effect increases expression of the gene product in the Drosophila cell at high copy number. Examples of expression systems useful for this method include, but are not limited to, the gene switch protocol and the Haig Keshishian RU486 method. The gene switch system, also known as the Gal80 system, works as follows: a temperature sensitive allele of Gal80 is combined with the usual gal4-UAS constructs: a gal4 driver, a tissue-specific reporter and a Gal80 cassette to repress Gal4 and stop its ectopic gene expression. It takes approximately 3-6 hours to get peak expression and then 15 hours to turn peak expression off, so if toxicity were a problem with any of these constructs, this can be a useful misexpression system. The Haig Keshishian system uses RU486, which is also referred to as a gene switch, and is similar to a Gal4 system developed using estrogen receptors, allowing steroid hormones to act as the activating switch. This system can be thought of as a modified Gal4 system, dependent on the presence of mifepristone (RU486). Transgenic lines express this modified Gal4 protein, which remains inactive until bound to mifepristone; it can then bind to UAS sequences as would a normal Gal4 molecule and initiate transcription. (Osterwalder et al (2001) PNAS 98(22): 12596-601.)

The Drosophila actin 5C gene promoter (B. J. Bond et al, 1986) is also a desirable promoter sequence. The actin 5C promoter is a constitutive promoter and does not require addition of metal. Therefore, it is better-suited for use in a large scale production system, like a perfusion system, than is the Drosophila metallothionein promoter. An additional advantage is that the absence of a high concentration of copper in the media maintains the cells in a healthier state for longer periods of time.

Examples of other known Drosophila promoters include, e.g., the inducible heatshock (Hsp70) and COPIA LTR promoters. The SV40 early promoter gives lower levels of expression than the Drosophila metallothionein promoter.

The transgene may be integrated into the fly genome in a manner that provides for direct or indirect expression activation by the promoter, i.e. in a manner that provides for either cis or trans activation of gene expression by the promoter. In other words, expression of the transgene may be mediated directly by the promoter, or through one or more transactivating agents. Where the transgene is under direct control of the promoter, i.e. the promoter regulates expression of the transgene in a cis fashion, the transgene is stably integrated into the genome of the fly at a site sufficiently proximal to the promoter and in frame with the promoter such that cis regulation by the promoter occurs.

In other embodiments where expression of the transgene is indirectly mediated by the endogenous promoter, the promoter controls expression of the transgene through one or more transactivating agents, usually one transactivating agent, i.e. an agent whose expression is directly controlled by the promoter and which binds to the region of the transgene in a manner sufficient to turn on expression of the transgene. Any convenient transactivator may be employed. The GAL4 transactivator system is an example of such a system.

The GAL4 encoding sequence can be stably integrated into the genome of the animal in a manner such that it is operatively linked to the endogenous promoter that provides expression in the appropriate location. The GAL4 system consists of the yeast transcriptional activator GAL4 and its target the upstream activating sequence (UAS) located within the P-element. Initially, GAL4 and UAS are in separate lines. The UAS is mobilized to generate new UAS insertion lines which remain silent until a source of GAL4 is made available. Under the control of a promoter, the expression of GAL4 is directed in a particular pattern. Specialized promoters can be used to drive expression of GAL4 in tissue and cell specific manners. The GAL4 containing line is then crossed to the UAS containing line. The UAS in the presence of GAL4 directs the expression of any genes adjacent to its insertion site. When the insertion site is located upstream from the coding region over- or ectopic expression occurs.

Flies of line 31-1 (also referred to as 1822), as disclosed in Brand & Perrimon, 1993 express GAL4 in this manner, and are known to those of skill in the art. The transgene is stably integrated into a different location of the genome, generally a random location in the genome, where the transgene is operatively linked to an upstream activator sequence, i.e. UAS sequence, to which GAL4 binds and turns on expression of the transgene. Transgenic flies having a UAS: GAL4 transactivation system are known to those of skill in the art and are described in Phelps & Brand, 1998.

In one embodiment, the subject transgenic flies are produced by: (1) generating two separate lines of transgenic flies: (a) a first line that expresses GAL4; and (b) a second line in which the transgene is stably integrated into the cell genome and is fused to a UAS domain; (2) crossing the two lines; and (3) screening the progeny for the desired phenotype, i.e. adult onset neurodegeneration. Each of the above steps are well known to those of skill in the art.

(2) Transgenes

(a) Neurodegenerative Transgenes

Neurodegenerative diseases or disorders according to the disclosed methods comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis. There are, in addition, a number of other diseases that can be modeled effectively in the fly via loss of function genetics, and that could be used in this screen: SMA (spinal motorneuronal atrophy) AD-HSP (autosomal dominant hereditary spastic paraplegia), dHMNII (distal hereditary motor neuropathy type II, schizophrenia (strophinl and dysbindin), depression (wolframin), ADOA (autosomal dominant optic atrophy, caused by mutations in dynamin), GDNF for Parkinson's disease (glial cell line derived neurotrophic factor).

Many neurodegenerative diseases have been linked to amyloidgenic peptides, and are therefore known as amyloidgenic diseases. Most of the amyloidgenic peptides cause diseases in the elderly and seem related to long term deposition of insoluble fibrils that lead to cellular toxicity by as yet unknown mechanisms. Examples of amyloidgenic diseases include, but are not limited to, Alzheimer's Disease, Parkinson's Disease, Huntington's Disease, massive bleeding strokes (CAA), as well as some forms of Diabetes, polycystic kidney disease, and cardiac amyloidosis.

Alzheimer's disease is related to the Amyloid precursor protein and its related peptides. Parkinson's Disease is related to human mutant alpha-synuclein. Huntington's Disease is related to huntingtin. Amyetropic lateral Sclerosis is related to Superoxide Dismutase 1 (SOD1). Progressive nuclearpalsyis related to alpha-syncuclein and microtubule associated tau protein. Corticobasal degeneration is related to tau and alpha-synuclein. Age-related macular degeneration is modeled by d-best (drosophila ortholog of bestrophin, implicated in juvenile onset macular degeneration). Motomeuron diseases are related to SMN 1, 2. Depression is related to wolframin. Schizophrenia is related to dysbindin.

Amyloidgenic diseases are generally characterized as being diverse, causing disease only in gain of function contexts (i.e., loss of gene function does not lead to amyloidgenic disease), and having causative agents that lack homology to one another. These causative agents are known as amyloidgenic peptides and are characterized by the presence of extensive amounts of beta sheet structure, staining by Congo Red and other Beta sheet intercalators, toxic conformiational states associated with the deposition of fibrillar material, and fibrils that develop from pre-fibrillar soluble states.

A large number of peptides are now recognized as being amyloidgenic. these include prions (SEQ ID NO: 1), Amyloid A (AA) (SEQ ID NO: 2), Amyloid Precursor Protein (APP) and its derivatives, IAPP (diabetes), TTR, Huntington, alpha-synuclein, polyglutamine expansion repeat proteins, such as SCA1 and Fragile X protein.

The protein encoded by SEQ ID NO: 1 is a membrane glycosylphosphatidylinositol-anchored glycoprotein that tends to aggregate into rod-like structures. The encoded protein contains a highly unstable region of five tandem octapeptide repeats. This gene is found on chromosome 20, approximately 20 kbp upstream of a gene which encodes a biochemically and structurally similar protein to the one encoded by this gene. Mutations in the repeat region as well as elsewhere in this gene have been associated with Creutzfeldt-Jakob disease, fatal familial insomnia, Gerstmann-Straussler disease, Huntington disease-like 1, and kuru. Two transcript variants encoding the same protein have been found for this gene. This sequence can be used to generate invertebrate embryo phenotypes which are useful with the methods disclosed herein.

The protein encoded by SEQ ID NO: 2 is Amyloid A, which is associated with Cerebral Amyloid Angiopathy. Cerebral Amyloid Angiopathy (CAA) is recognized as a major cause of strokes in the elderly. The term CAA refers to precipitates of Amyloid A protein (an amyloidgenic peptide) in blood vessels that allow blood to leak out and cause hemorrhagic (bleeding) strokes. This sequence can be used to generate invertebrate embryo phenotypes which are useful with the methods disclosed herein.

Other sequences that can be used to generate fly phenotypes which are useful with the methods disclosed herein include, but are not limited to, those related to the Amyloid Precursor Protein (APP) and fragments and derivatives thereof. Examples of those fragments and derivatives are known in the art and include APPL, CT100, β-CTF, α-CTF, γ-CTF, Abeta₁₋₄₀, Abeta₁₋₄₂, Abetass₁₋₄₀, and Abetass₁₋₄₂ All sequences referred to herein can be found in GenBank, which sequences are herein incorporated by reference in their entirety. The extracellular portion of APP is cleaved by either α-secretase or β-secretase. This appears to be in response to a signaling event. γ-secretase is then cleaved within the membrane by a process known as regulated intramembraneous proteolyis (RIP), thereby generating the apparently toxic Abeta fragment. There is slippage in the γ-secretase cleavage site. Cleavage of the 100 amino acid C-terminal fragment (CT100) can occur at residues 40, 42, and 57. The sequences of these amyloigenic peptides are as follows

SEQ ID NO: 3:
DAEFRHDSGYEVHHQKLVFFAEDVGSNKGAIIGLMV (Abeta₁₋₄₀)
SEQ ID NO: 4:
DAEFRHDSGYEVHHQKLVFFAEDVGSNKGAIIGLMVIA (Abeta₁₋₄₂)
SEQ ID NO: 5:
DAEFRHDSGYEVHHQKLVFFAEDVGSNKGAIIGLMVIATVIVITLVMLKK
QYTS (Abeta₁₋₅₇)
SEQ ID NO: 6:
DAEFRHDSGYEVHHQKLVFFAEDVGSNKGAIIGLMVIATVIVITLVMLKK
QYTSIHHGVVEVDAAVTPEERHLSKMQQNGYENPTYKFFEQMGNCOO
(CT100) (also known as CT99)

As described above, transgenic invertebrate strains can be made which express genes relating to a neurodegenerative diseases, and specifically to amyloidgenic diseases. Examples of such transgenes include, but are not limited to, UAS-Abeta₁₋₄₀ UAS-ssAbeta₁₋₄₀ UAS-Abeta₁₋₄₂, UAS-ssAbeta₁₋₄₂, UAS-CT100, UAS-ssCT100, UAS-A53T α-synuclein, UAS-α-synuclein, UAS-wolframin, and UAS-Flywolf. Abeta₁₋₄₀ and Abeta₁₋₄₂ are expressed throughout the CNS with pan-neuronal drivers (Example 2). These transgenes can comprise a gene coding for a vertebrate amyloid precursor protein (APP), or a fragment, or a derivative or a variant thereof, a gene coding for a vertebrate α-secretase, for example. Each of these sequences is identified from published sequences, is attached (ligated) to sequences containing UAS binding sites within a vector containing p-element insertion sites. These vectors encoding the assembled transgenes are then injected into Drosophila embryos for the generation of transgenic fly strains. Abeta transgenes relate to Alzheimer's disease, Alpha-synuclein transgenes relate to Parkinson's disease, wolframin and Flywolf are related to a model of depression. Specifically, 12 gain of function mutant lines related to Parksinson's Disease have been generated. These are UAS-A53T alpha synuclein lines. These were generated by Genetic Services, Inc. A sequence for human A53T alpha synuclein was cloned into the pUAST vector, purified, checked for sequence and orientation, and sent to Genetic Services for injection. The twelve lines have been balanced and mapped. Chromosomal insertion sites are as follows: one insert was on the X, three were on the Second Chromosome and five were on the third chromosome. Of the 12 lines, most were homozygous viable, including SZ8. It has been misexpressed and examined for embryonic ventral nerve cords for loss of dopaminergic neurons, and stained with Drosophila TH antibodies and with TUNEL staining (Moelcular Probes).

Regarding depression, it has been demonstrated that the mood disorders associated with Wolfram's Syndrome are correlated with disruptions of the small cell layers of the cortex and show that the subcellular pathology is similar in flies and humans (Example 6).

In one embodiment, the embryo also expresses a gene coding for a protein having γ-secretase activity. The gene can code for a presenilin, which can be a vertebrate or an invertebrate gene. It can be a human gene, in particular presenilin-1 or presenilin-2, or fragments, derivatives or variants thereof, for instance known human mutated versions thereof that have been implicated in the early-onset, familiar form of Alzheimer's disease. However, it might also be preferred that the animal of the instant invention expresses the endogenous gene coding for presenilin, or a fragment, or a derivative, or a variant thereof. In one embodiment of the invention, a gene coding for a protein having γ-secretase activity, in particular a gene coding for presenilin, is over-expressed or mis-expressed. The expression can be ectopic, which means that said genes are expressed in a tissue or cell or at a defined developmental stage of the invertebrate animal where they are not normally expressed.

The UAS-Gal4 system was used to misexpress the A53T alpha synuclein gene in dopaminergic neurons via the dopadecarboxylase gal4 (Ddc-gal4) line. A53T had been identified in Mediterranean families as a strong link to early onset Parksinson's Disease. The A30P point mutated form of alpha synuclein (identified in a German family as being strongly correlated with early onset Parkinson's disease) was misexpressed in dopaminergic neurons via the Ddc-gal4 driver. A specific age-related neurodegeneration, motordefects that mimicked those observed in human PD patients, and the development of Lewy Body like neurofibrillary tangles in dopaminergic neurons was found. It has been found that these drugs ameliorated the symptoms in flies in the same dosage and efficacy ranges as observed in humans.

(b) Functional Nucleic Acids

Another set of molecules that can make up the transgene are functional nucleic acids.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. These functional nucleic acids can be used in the screening methods disclosed herein. The functional nucleic acid molecules can act as affectors, inhibitors, modulators, and stimulators of a specific activity possessed by a target molecule, or the functional nucleic acid molecules can possess a de novo activity independent of any other molecules.

RNA interference (RNAi) and small interfering RNA (SiRNA) molecules are examples of functional nucleic acids and can be used with the screening methods disclosed herein. It is thought that RNAi involves a two-step mechanism for RNA interference (RNAi): an initiation step and an effector step. For example, in the first step, input double-stranded (ds) RNA is processed into small fragments (siRNA), such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that an act in RNAi.

RNAi has been shown to work in a number of cells, including mammalian and invertebrate cells. In certain embodiments the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long.

Methods of RNAi and SiRNA are described in detail in Hannon et al. (2002), RNA Interference, Nature 418:244-250; Brummelkamp et al. (2002), A System for Stable Expression of Short Interfering RNAs in Mammalian Cells, Science 296:550-508; Paul et al. (2002), Effective expression of small interfering RNA in human cells, Nature Biotechnology 20: 505-508, which are each incorporated by reference in their entirety for methods of RNAi and SiRNA and for designing and testing various oligos useful therein.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of United States patents: U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of United States patents: U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following United States patents: U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following United States patents: U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following United States patents: U.S. Pat. Nos. 5,595,873 and 5,652,107). There are also a number of ribozymes that are not found in natural systems, but which have been engineered to catalyze specific reactions de novo (for example, but not limited to the following United States patents: U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of United States patents: U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of United States patents: U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of United States patents: U.S. Pat. Nos. 5,168,053, 5,624,824, 5,683,873, 5,728,521, 5,869,248, and 5,877,162.

(3) Other Possible Components for Transgene Constructs

A desirable gene expression unit or expression vector for the protein of interest can also be constructed by fusing the protein coding sequence to a desirable signal sequence. The signal sequence functions to direct secretion of the protein from the host cell. Such a signal sequence may be derived from the sequence of tissue plasminogen activator (tPA). Other available signal sequences include, e.g., those derived from Herpes Simplex virus gene HSV-IgD (Lasky et al, Science, 233:209-212 1986).

The DNA coding sequence can also be followed by a polyadenylation (poly A) region, such as an SV40 early poly A region. The poly A region which functions in the polyadenylation of RNA transcripts appears to play a role in stabilizing transcription. A similar poly A region can be derived from a variety of genes in which it is naturally present. This region can also be modified to alter its sequence provided that polyadenylation and transcript stabilization functions are not significantly adversely affected.

The recombinant DNA molecule can also carry a genetic selection marker, as well as the protein gene functions. The selection marker can be any gene or genes which cause a readily detectable phenotypic change in a transfected host cell. Such phenotypic change can be, for example, drug resistance, such as the gene for hygromycin B resistance (i.e., hygromycin B phosphotransferase).

Alternatively, a selection system using the drug methotrexate, and prokaryotic dihydrofolate reductase (DHFR) gene, can be used with invertebrate cells. The endogenous eukaryotic DHFR of the cells is inhibited by methotrexate. Therefore, by transfecting the cells with a plasmid containing the prokaryotic DHFR which is insensitive to methotrexate and selecting with methotrexate, only cells transfected with and expressing the prokaryotic DHFR will survive. Unlike methotrexate, selection of transformed mammalian and bacterial cells, in the Drosophila system, methotrexate can be used to initially high-copy number transfectants. Only cells which have incorporated the protective prokaryotic DHFR gene will survive. Concomitantly, these cells have the gene expression unit of interest.

(4) Analysis of the Transgene

Transcription and expression of the heterologous protein coding sequences can be monitored. For example, Southern blot analysis can be used to determine copy number of the gp120 gene. Northern blot analysis provides information regarding the size of the transcribed gene sequence. The level of transcription can also be quantitated. Expression of the selected protein in the recombinant cells can be further verified through Western blot analysis, for example. The transcripts and inserts can also be monitored using the polymerase chain reaction or other amplification procedures used to amplify specific sequences, understood in the art. Furthermore, any nucleic acid detection assay, such as DNA chip technology can also be used to monitor the presence of specific nucleic acids.

(5) Strains

Any Drosophila cell line can be used with the methods described herein. Table 2, for example, provides a list of cell lines that can be used with the methods described herein. Examples of Drosophila cell lines can also be found at The FlyBase Consortium (The FlyBase database of the Drosophila genome projects and community literature. 2003, Nucleic Acids Research 31:172-175. http://flybase.org/).

It will be readily apparent to those individuals skilled in the art that any genetically-modified Drosophila strains can be used with the methods disclosed herein. Preferably, the Drosophila strain has an easily monitored phenotype which is detectably altered in response to the modification of genes related to disease pathways. Appropriate disease pathways include, but are not limited to, neurodegenerative disease signaling pathways such as Parkinson's and Alzheimer's; singaling pathways controlled by the Ras proto-oncogene; the WNT tumor suppressor gene; Rb (retinoblastoma tumor suppressor gene); HH (hedgehog development regulator) or the HH vertebrate homolog SHH (sonic hedgehog developmental regulator); activated protein kinase B (PKB/AKT); insulin receptor; insulin receptor substrates (IRS); c-src proto-oncogene; c-Jun proto-oncogene; c-myc proto-oncogene; p53; Janus kinases (JAK/STAT pathway); nitric oxide (NO); calmodulin; cAMP dependent protein kinase (PKA); Ca²⁺ dependent protein kinase (PKC); growth factors such as GH, TGF, PDGF and the like; receptor tyrosine kinases (RTKs); interferons (IFN); lipid metabolites; steroid hormones; phosphatidylinositol; G-protein coupled receptors; c-abl proto-oncogene; TGF, and Smad gene family members; interleukins; GTPases; and ionophores.

8. Insects

Arthropods include Crustacea, which are things like prawns, crabs and woodlice; Myriapoda, which are centipedes, millipedes and such; Chelicerata (Arachnida), which are spiders, scorpions and harvestmen etc., and Uniramia (Insecta), which are things like beetles, bees and flies.

Insects are found in the phylum Arthorpoda, Subphylum Insecta (also often called a class), Class Hexapoda, and Subclasses Apterygota, Exopterygota, and Endopterygota. The Apterygota includes the orders Protura, Collembola (Springtails), Thysanura (Silverfish), Diplura (Two Pronged Bristle-tails). The Exopterygota includes the orders Ephemeroptera (Mayflies), Odonata (Dragonflies), Plecoptera (Stoneflies), Grylloblatodea, Orthoptera, Phasmida (Stick-Insects), Dermaptera (Earwigs), Embioptera (Web Spinners), Dictyoptera (Cockroaches and Mantids), Isoptera (Termites), Zoraptera, Psocoptera (Bark and Book Lice), Mallophaga (Biting Lice), Siphunculata (Sucking Lice), Hemiptera (True Bugs) Thysanoptera, The Endopterygota includes the orders Neuropter (Lacewings), Coleoptera (Beetles), Strepsiptera (Stylops), Mecoptera (Scorpionflies), Siphonaptera (Fleas), Diptera (True Flies which are unusual in that they only have one pair of functional wings. The other pair is reduced to a pair of knoblike organs, called halteres, which play a part in stabilizing these insects during flight. True flies include house flies and bluebottles, mosquitoes, horseflies, midges, and antler-headed flies. Also included are Lepidoptera (Butterflies and Moths), Trichoptera (Caddis Flies), and Hymenoptera (Ants Bees and Wasps).

An example of Drosophila species that are useful include, but are not limited to, D. pseudoobscura, D. virilis, D. hydei, D. mauritiana, D. simulans, D. subobscura, and D. odysseus.

9. Sorting of Embryos

Drosophila lethal mutations are maintained over a balancer chromosome. Balancer chromosomes are multiply inverted chromosomes, used to minimize recombination events, that carry lethal mutations so that homozygous balancer embryos die and that also carry dominant markers so that the presence of a balancer chromosome can be readily identified. Embryos produced by parents with the genotype mutation/balancer produce offspring of the following genotypes: homozygous mutant embryos (mutation/mutation), heterozygous embryos (mutation/balancer) and homozygous balancer embryos (balancer/balancer). Only the heterozygous mutant/balancer embryos can develop into adults and so maintain a stable stock. However, for any embryo collection from these parents, only about 25% of the embryos contain the homozygous mutation of interest. Using a GFP marker gene on the balancer chromosome it is possible to identify this minority population of mutant embryos from their balancer containing siblings, in living embryos.

In one embodiment, embryos can be sorted using an automated system. An automated sorter can be used to achieve two important goals: 1) heterozygous embryos (which have normal phenotypes in comparison to homozygous siblings) can be separated from homozygous embryos; 2) homozygous embryos can be precisely aliquoted. The principle of automated sorting of living Drosophila embryos can be demonstrated using flow cytometry technology. The Union Biometrica COPAS SELECTsystem™ is an example of such a system. It can separate Drosophila embryos expressing genes of interest from initially large populations of embryos.

The COPAS SELECT can sort embryos exhibiting genetic expression by measuring the fluorescence intensity of the protein reporter expressed during early embryo development. For example, a population of embryos containing GFP-tagged balancer chromosomes such as CyO armadillo-GFP, CyOactinGFP, or CyOKruppelGFP. The same drivers direct GFP expression on the third chromosome balancers (TM3 and TM6) can be used. Those embryos not expressing GFP can be selected and sorted from the original population with a high level of accuracy and purity. Embryos can also be isolated that display specific morphological characteristics. For example, an embryo can be sorted out of a population based on its size. The relative size of each embryo can also be analyzed. This information may be used in conjunction with fluorescence intensity, to isolate unique embryos.

In one embodiment, Drosophila embryos are diluted using an appropriate solution such as “Liquid Gold”. The embryo concentration can then be checked by extracting a known volume of liquid out from the preparation and by counting the amount of embryos present. Multiple aliquots of the solution can be extracted in order to confirm the recommended concentration of embryos. The diluted embryos can then be analyzed. In the COPAS SELECTsystem™, two parameters of optical characteristics, Green Fluorescence Intensity (FLU1) and Extinction (EXT), can be used to initially analyze the population. EXT is a measure of optical density and FLU is a measure of fluorescence including autofluorescence, fluorescent protein expression, or fluorescent markers.

All of the embryos analyzed during the experiment can be displayed simultaneously. The desired embryos are then isolated from the whole population. The sort region can be defined using FLU1 (green fluorescence intensity) and FLU2 (red fluorescence intensity) as the two parameters. A collection vial can then be used to gather the embryos of interest. When using these methods, a greater than 95% purity when 15 to 25% of the total population was selected can be observed (COPAS SELECTsystem Application Guide S01, http://www.unionbio.com/applications/app_notes/d_melan_files/DrosophilaANS01.pdf).

10. Analysis/Readout Assay

After sorting, Drosophila embryos can be used in the screening methods disclosed herein. Screening optionally takes place in multi-well plates. Multi-well plates are standard in the art and come in a variety of sizes and shapes. For example, the multi-well plate can be 24, 48, or 96 well plates. Such screening assays can be automated or further modified for high throughput analysis.

Chemical compounds are then delivered to embryos in the well plates. For high throughput screening, each well can include numerous test components. An assay of phenotypic improvement is then carried out using seed fill algorithms. If a positive reaction is detected in a well, the screening is repeated with one of the test compounds contained in a single well.

Seed fill algorithms can be used in the following manner. Embryos develop in the drug, absorb the drug, and are then scored for their reaction to the drug. The reaction can be revealed, for instance, by the expression of Red/Blue/Yellow Fluorescent Protein. A plate reader can use a number of extra features to minimize error in pattern detection, and image data can then be collected, transferred via high speed data storage systems, and then analyzed. The initial analysis can be performed by computer, using a seed fill algorithm, which determines whether a data point should be kept for subsequent study or whether it should be eliminated. Example 3 describes one embodiment of a seed fill algorithm. For example, the plates can be black-welled plates available from a number of suppliers. These are flat-bottomed plates with black walls to minimize fluorescent bleed-through, effectively isolating the contents of each well for optical analysis. Plates are automatically loaded onto the plate reader. Internal standards direct the focal plane to a particular focal plane within the embryo. Such standards include a number of specific markers such as vital dyes which are membrane specific. Because the axons of the embryonic CNS lie on the dorsal surface of the CNS, the highest concentration of this dye (in the violet visible light range) directs the focal plane to the dorsal surface of the CNS. Focusing 35 microns below or above the center of this intense purple stain directs the reader to photograph at the level of dopaminergic neurons in the vnc nerve cord, for example. Such specific landmarks can be determined a priori for each disease model of interest. Z-series stacks can be acquired, or single images and these comprise the data sets for each well. These data sets are subjected to analysis by seed fill algorithms.

Seed fill algorithms are commonly used algorithms in which the computer performs a pixel by pixel analysis of intensity levels. Since fluorescent proteins never vary in intensity, seed fill algorithms can analyze them with consistency and reproducibility. changes in the number of contiguous pixels meeting the intensity requirement are scored as positive or negative hits, depending on the assay and disease being assayed. For example, for the Alzheimer's model, amelioration of the phenotype can be scored as increased numbers of contiguous pixels. For a Parkinson's model in which distance between intensity loci are measured, decreased readouts can be scored as hits.

11. Exemplary Embodiments

Specifically, disclosed are methods of screening a candidate compound for its effect on a disease. These methods include administering the compound to an invertebrate animal embryo and assaying the effect of the compound on the embryo. These methods also include comparing phenotypes of the transgenic embryo treated with the compound to a transgenic invertebrate embryo not treated with the compound, wherein a difference in the phenotypes is indicative of the alleviating activity of the candidate compound.

These methods also include introducing a nucleic acid into an invertebrate animal, wherein the nucleic acid encodes a peptide associated with a phenotype of the disease, expressing the phenotype in an embryo of the invertebrate animal, aliquoting the embryos of into wells of a plate, delivering a test compound to the well of the plate, and screening for a change in the phenotype associated with the disease, a change indicating a compound with an effect upon the disease.

Also disclosed are methods of screening a compound for its effect on an invertebrate embryo. These methods include dechorionating the embryo, incubating the dechorionated embryo with the compound and a cell perforating agent such as DMSO, and assaying the effect of the compound on the embryo. Also disclosed are high through put mechanisms utilizing the above screening methods.

Disclosed herein are a methods of manipulating an invertebrate animal embryo, comprising delivering a composition to the embryo. Also disclosed is a method of introducing a compound into an invertebrate embryo, comprising administering to the embryo an effective amount of an agent that facilitates cellular uptake, such as DMSO.

Disclosed are methods of screening for a test compound with an effect on a phenotype associated with a neurodegenerative disease. These methods can include, for example, the steps of placing transgenic embryos in a read out assay, delivering a test compound to the read out assay, and scoring the embryos for the ability to extend axons across segment boundaries, said ability indicative of a test compound with an effect on a phenotype associated with a neurodegenerative disease. The transgenic embryos can express a membrane-tagged fluorescent protein, for example.

Also disclosed are methods for evaluating the phenotypes of invertebrate animal embryos. Disclosed herein are a methods of manipulating an invertebrate animal embryo, comprising delivering a composition to the embryo. Also disclosed is a method of introducing a compound into an invertebrate embryo, comprising administering to the embryo an effective amount of an agent that facilitates cellular uptake, such as DMSO

Also disclosed are methods of making a genetic assay. These methods include introducing a nucleic acid into an invertebrate animal, wherein the nucleic acid encodes a peptide associated with a phenotype of a disease, expressing the phenotype in an embryo of the invertebrate animal, aliquoting the embryos into wells of a plate, and delivering a test compound to the well of the plate.

Specifically, disclosed are methods of making a genetic assay for a neurodegenerative disease. Included in these methods are, for example, the steps of introducing a nucleic acid into an invertebrate animal, wherein the nucleic acid encodes a peptide associated with a phenotype of the neurodegenerative disease, introducing a membrane-bound fluorescent protein in to the invertebrate animal, introducing a UAS-Gal4 system into the invertebrate animal, and expressing the phenotype in an embryo of the invertebrate animal. The peptide associated with a phenotype of the neurodegenerative disease can be, for example, an amyloidgenic peptide, such as Abeta₁₋₄₂ or Abeta₁₋₄₀. A signal sequence can also be introduced into the invertebrate animal.

Disclosed are methods of scoring a plate. Such methods can include the steps of placing transgenic embryos expressing a phenotype associated with a disease in a read out assay, delivering a compound to the read out assay, and using a seed fill algorithm to score the embryos, wherein the algorithm evaluates contiguous lines of pixels, wherein a compound that ameliorates the phenotype gives a higher score in comparison to a control embryo.

Also disclosed are kits for use in screening compounds. The kit can include, for example, transgenic invertebrate animal embryos and a suitable container. Also disclosed is an embryo sorter capable of selecting homozygous embryos, as well as a read out assay useful in a high throughput screen of invertebrate embryos. An example of a read out assay comprises using the following algorithm: 1) images are collected at 512×512 sizes; 2) each of the resulting 262,144 pixels is evaluated on a typical 1-256 grey scale. Because the fluorescent proteins do not vary in intensity, there is no variability from prep to prep or among embryos within a prep. 3) Each pixel is analyzed for its intensity. If it meets the threshold value (e.g. 200), it is assigned a value of 1. If it fails to meet that intensity level, it is assigned a value of 0. 4) Each pixel is evaluated in a pairwise fashion, as follows: Each pixel is surrounded by 8 other pixels. Consider a number pad of the type found on telephones or calculators. If the number 5, surrounded by the numbers/pixels 7, 8, 9, 6, 3, 2, 1, and 4 is found to be adjacent to pixels sufficiently bright to meet the cutoff at positions 2 and 8, the algorithm moves in both of those vertical directions until it encounters a pixel that fails to meet that criterion. The logic string determines a directionality. So that if 8 and 2 are found to meet the intensity criteria, then future consideration is given only to pixels in that column and branching in non-linear directions is discounted. However, unless a directionality is specified, all linear clusters will be counted—including horizontal ones; branched clusters are never considered. 5) The longest strings are counted and retained as the data point for that image, and then the computer moves on to the next field of view.

For example, the read out for the Alzheimer's Disease phenotype is that axons extend further in the presence of drugs which suppress the ability of the Abeta peptides to stall them. So if the Abeta peptide poisons axonal transport, then molecules that improve axonal transport by for example, upregulating the expression or function of kinesins (the motors that bind cargo to microtubules for axonal transport to the synapse) are be expected to improve the ability of axons to extend in these genetic backgrounds. The abeta peptide is still there, but the drug is directly interfering with its pathological properties. In the case of Parksinon's Disease, in which A53T alpha-synculein misexpression in dopaminergic neurons leads to the loss or shrinkage of dopaminergic neurons, the seed fill algorithm is designed to either determine absolute size of dopaminergic neurons, or the distance between dopaminergic neurons. Drugs that interfere with A53T alpha synuclein toxicity, perhaps by upregulating degradation of even insoluble peptides by the proteasome, or by enhancing the ability of the mitochondria to tolerate the oxidative stress putatively incurred in alpha-synuclein overload would be recovered as positive “hits”. Also recovered as hits in a Parkinson's disease screen are molecules that inhibit dopamine synthesis, since the neurotransmitter, dopamine, actually stabilizes the amyloidgenic fibrils derived from mutated forms of alpha-synuclein (like A53T Alpha synuclein); in other words, the reason dopaminergic neurons are susceptible to the accumulation of alpha synuclein plaques can be because dopamine actually reinforces the plaques.

Also disclosed are methods of making an invertebrate animal embryo useful for screening compounds. For example, the embryo can be genetically modified. A recombinant DNA molecule or vector containing a heterologous protein gene expression unit can be used to transfect invertebrate cells (U.S. Pat. Nos. 4,670,388 and 5,550,043, herein incorporated by reference in their entirety.) A gene expression unit can contain a DNA coding sequence for a selected protein or for a derivative thereof. Such derivatives can be obtained by manipulation of the gene sequence using traditional genetic engineering techniques, e.g., mutagenesis, restriction endonuclease treatment, ligation of other gene sequences including synthetic sequences and the like (T. Maniatis et al, Molecular Cloning, A Laboratory Manual., Cold Spring Harbor Laboratory, Cold Spring Harbor, N.Y. (1982).

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the amino acids are discussed, specifically contemplated is each and every combination and permutation of the transgene and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Compositions Used in Screening

The ability to introduce chemical compounds into Drosophila embryos is the basis for screening methods that provide four major advances for high-throughput screens of chemical compounds: it improves the accuracy and speed of template identification; it allows for the simultaneous evaluation of compound efficacy and organism-wide compound toxicity; it allows high throughput screening approaches in genetic model systems in which human diseases can be modeled with unparalleled success (Feany and Bender, 2000); and it is the most cost-effective approach for compound screening to date, requiring on the order of 1/1000^{th} the drug typically required in mammalian cell-based assays. The ease of secondary assays to validate targets is an additional advantage.

The composition can be any compound of a suitable size for uptake by the embryo. For example, the composition can follow the guidelines of “Lipinski's Rule of Five.” (Lipinski, 1997). Lipinski's Rule of Five is particularly useful when the goals of compound design are (i) to have less than 5 hydrogen donors, (ii) less than 10 hydrogen bond acceptors, (iii) molecular weight of less than 500 Daltons and (iv) the log of the partition coefficient, P (where P the concentration of the compound in water divided by the concentration of the compound in 1 octanol) is less than 5. The Lipinski Rule of Five is a useful guideline, however, the composition is not limited to these parameters.

A wide variety of small molecular weight compounds can be used in the screening methods disclosed herein. Such compounds include, but are not limited to, any compositions which are being tested for drug discovery or development. Such compounds include, but are not limited to, nucleic acids including functional nucleic acids, amino acids including peptides and proteins and fragments thereof, and various other chemical compounds. Compounds can be aqueous- or lipid-soluble. Compounds can be delivered individually to invertebrate animal embryos or may be delivered to as one of a plurality of different chemical compounds. Compounds can be dissolved or suspended within solution, or affixed to a solid-support. Solid supports may include, but are not limited to, insoluble polymer beads or a polymeric matrix coated with one or a plurality of individual compounds, or with combinatorial chemistries. Dosages and volumes which are administered to the Drosophila embryo can be varied so as to optimize dosages for further studies or to rank compounds as to their toxicity and/or potency. Information resulting from variations in conditions can be used to prioritize chemicals for further study, to delineate the relative toxicities of structurally related chemicals, and/or to identify the proper dose range for subsequent toxicity studies (see e.g., Harris, et al., Fundatn. Appl. Toxicol. 19:186-196).

The carbon-carbon backbone of the compounds can be saturated or unsaturated, cyclic or linear. These aforementioned compounds include, but are not limited to, carbohydrates, polyalcohols (e.g., ethylene glycol and glycerol) and polyphenols (e.g., hydroquinones and tetracylines). Carbohydrate- and polysaccharide-transformed compounds are defined herein so as to include all chemical moieties possessing a saccharide unit or which are transformed from a saccharide. These compounds can also include glycopeptides, glycolipids and other biopolymers (or biomacromolecules) containing saccharides, either in their entirety or as part of the molecular framework. The term carbohydrates merely represent a portion of a much larger family of polyhydroxylated organic compounds. In addition, carbohydrated/polyhydroxylated organic compounds include, but are not limited to: monomeric acyclic compounds (e.g., ethylene glycol, glycerol and 1,2,3-trihydroxy pentane); polymeric acyclic compounds (e.g., di- or tri-ethylene diglycol; monomeric cyclic compounds (e.g., inositol and 1,2,3-trihydroxycyclopentane); polymeric cyclic compounds (e.g., di-inositol); polymeric and monomeric unsaturated compounds (e.g., tetrahydroxy-1,4-quinone) and polyphenols (e.g., tetracyclines) and derivatives, analogs and fragments thereof.

With respect to the generation of small molecular weight compound libraries, the combination of biochemical diversity is often synergistic with the metabolic diversity obtained from the in vivo production of “natural products”. Collections of starting compounds, for example peptides, can be administered to cultures of microorganisms. In accord, each microbial strain may potentially create numerous modified peptides or peptide byproducts, thus generating a “metabolite library”. Because each of these aforementioned cultures can contain a very complex mixture of metabolites, a highly efficacious method of screening is required (i.e., HTS). An aliquot of the library is incubated with each of the many strains typical of a microorganism fermentation screening program, and the media screened utilizing an HTS-based assay. Furthermore, natural product diversity can be screened by creating a mixture of combinatorially-tagged liposomes; wherein each liposome preferably encapsulates only one member or a simple mixture of a natural product compound library. The libraries which are generated by the methodologies disclosed herein may be screened for any biological activity known within the art. These include, but are not limited to: anti-microbial activity, anti-tumor activity, enzyme inhibiting activity, receptor binding, growth promotion activity, and in vitro and in vivo tests for biological responses. Compounds may be based on naturally occurring extracellular or intracellular signaling molecules or their derivatives or the like (see, e.g., Alberts, et al., 1989. “Chapter 12: Cell Signaling.” 2nd Edition. Garland Publishing, Inc., New York, N.Y., pp. 681-726).

2. General Composition Parameters or Characteristics

a) Nucleic Acids

There are a variety of molecules disclosed herein that are nucleic acid based, including for example the neurodegenerative transgenes disclosed herein, as well as those nucleic acids that can be used in the screening methods disclosed herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to, for example, Abeta₁₋₄₀ and Abeta₁₋₄₂ as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

(4) Homology/Identity

It is understood that one way to define any known variants and derivatives or those that might arise, of the disclosed genes and proteins herein is through defining the variants and derivatives in terms of homology to specific known sequences. For example SEQ ID NO: 7 sets forth a particular sequence of a membrane glycosylphosphatidylinositol-anchored glycoprotein, and SEQ ID NO: see note above sets forth a particular sequence of the protein encoded by SEQ ID NO: 8, an Amyloid A protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman. and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

b) Peptides

(1) Protein Variants

As discussed herein there are numerous variants of amyloidgenic peptides, for example, that are known and herein contemplated. In addition to the known functional variants herein described, for example those related to the Amyloid Precursor Protein such as APPL, CT100, β-CTF, α-CTF, γ-CTF, Abeta₁₋₄₀, Abeta₁₋₄₂, Abetass₁₋₄₀, and Abetass₁₋₄₂, there are derivatives of APP which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 3 and 4 and are referred to as conservative substitutions.

TABLE 3
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 4
Amino Acid Substitutions
Original Residue Exemplary Conservative
Substitutions, others are known in the art.

Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is understood that for a mutation all of the nucleic acid sequences that encode a particular derivative are also disclosed. It is also understood that while no amino acid sequence indicates what particular DNA sequence encodes that protein within an organism, where particular variants of a disclosed protein are disclosed herein, the known nucleic acid sequence that encodes that protein in the particular location from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

c) Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with an invertebrate embryo in the screening methods disclosed herein.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable-immune responses when administered to humans. Therefore, the use of human or humanized antibodies in the methods serves to lessen the chance that an antibody administered to a human will evoke an undesirable immune response.

(2) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

d) Pharmaceutical Carriers

The compositions can also be used in the screening methods disclosed herein in conjunction with a pharmaceutically acceptable carrier. Alternatively, pharmaceutical carriers themselves can be tested by the screening methods disclosed herein. By “pharmaceutically acceptable” is meant a material that is not biologically or otherwise undesirable, i.e., the material may be administered to a subject, along with a nucleic acid or vector, without causing any undesirable biological effects or interacting in a deleterious manner with any of the other components of the pharmaceutical composition in which it is contained. The carrier would naturally be selected to minimize any degradation of the active ingredient as would be well known to one of skill in the art. The compositions may, for example, be in solution or suspension (i.e. incorporated into microparticles or liposomes).

Suitable carriers and their formulations are described in Remington: The Science and Practice of Pharmacy (19th ed.) ed. A. R. Gennaro, Mack Publishing Company, Easton, Pa. 1995. For example, an appropriate amount of a pharmaceutically-acceptable salt can be used in the formulation to render the formulation isotonic, and its effect on the invertebrate embryos can also be measured using the screening methods disclosed herein. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

3. Compositions Identified by Screening and Combinatorial Methods

Disclosed are the compositions that are identified through combinatorial techniques or screening techniques, as disclosed herein, in which the compositions are used in a combinatorial or screening protocol. It is understood that when using the disclosed compositions in combinatorial techniques or screening methods, molecules, such as macromolecular molecules, will be identified that have particular desired properties such as inhibition or stimulation or the transgenic embryo. Thus, the products identified using the combinatorial or screening approaches that involve the disclosed compositions are also considered herein disclosed.

It is understood that the disclosed methods for identifying molecules that have a desired effect upon the invertebrate embryo can be performed using high through put means disclosed herein.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference). These peptides can be used in the invertebrate embryo screening methods disclosed herein.

A preferred method for isolating proteins that have a given function, which can be used in the screening methods disclosed herein, is described by Roberts and Szostak, 1997. This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)). These compositions can then be used in the screening methods disclosed herein.

Another preferred method for combinatorial methods designed to isolate peptides for use in the screening methods herein are described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a neurodegenerative associated peptide, is attached to a DNA binding domain of a transcriptional activation protein, such as Gal4. By performing the two-hybrid technique on this type of system, molecules associated with neurodegenerative diseases, for example, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which are well known to those of skill in the art. These combinatorial libraries can then be used with the invertebrate embryo screening methods disclosed herein. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

4. Computer Assisted Drug Design

The compositions for use with the screening methods described herein can be used as targets for any molecular modeling technique to identify either the structure of another composition or to identify potential or actual molecules, such as small molecules, which interact in a desired way with the invertebrate embryos disclosed herein. The nucleic acids, peptides, and related molecules disclosed herein can be used as targets in any molecular modeling program or approach.

After a composition has been found to interact in a desired way with an invertebrate embryo, the composition can be used in modeling techniques. Molecules, such as macromolecular molecules, will be identified that have particular desired properties such as inhibition or stimulation or the target molecule's function. The molecules identified and isolated when using the disclosed methods are also disclosed. Thus, the products produced using the molecular modeling approaches that involve the disclosed screening methods, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

5. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein. Also disclosed are computer readable mediums comprising the sequences and information regarding the sequences set forth herein.

6. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include transgenic invertebrate animal embryos and a suitable container. The kit can also include combinatorial libraries of small molecules. The it can also include an embryo sorter. The kit can also include well plates for screening molecules.

7. Methods of Making the Compositions

The compositions disclosed herein and the compositions necessary to perform the disclosed methods can be made using any method known to those of skill in the art for that particular reagent or compound unless otherwise specifically noted.

a) Nucleic Acid Synthesis

For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

b) Peptide Synthesis

One method of producing proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

c) Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 1 or 2 for example, and a sequence controlling the expression of the nucleic acid. Such sequences controlling the expression of the nucleic acid are disclosed herein.

Also disclose are nucleic acid molecules produced by the process comprising linking in an operative way a nucleic acid molecule comprising a sequence having 80% identity to a sequence set forth in, for example, SEQ ID NO: 1 or 2, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth in SEQ ID NO: 1 or 2 and a sequence controlling the expression of the nucleic acid.

Disclosed are nucleic acid molecules produced by the process comprising linking in an operative way a nucleic acid molecule comprising a sequence encoding a peptide set forth in SEQ ID NO: 7 or 8 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO: 7 or 8 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO: 7 or 8, wherein any change from the sequence are conservative changes and a sequence controlling an expression of the nucleic acid molecule.

Disclosed are cells, including invertebrate embryos, produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals, including invertebrate embryos, produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Delivery of Chemical Compounds to Living Drosophila Embryos

Drosophila embryos are collected at 30 minutes prior to development. They are dechorionated by soaking in 100% bleach for 2 minutes. After extensive rinsing, embryos are transferred to 96 well plates containing chemical compounds at various concentrations, in 1% DMSO. Embryos are allowed to develop at 18 degrees Centigrade and assayed accordingly.

Initial studies examined the toxicity of DMSO in Drosophila embryos. Dose-response curves revealed that embryonic development proceeds normally to eclosion in concentrations of DMSO as high as 5% v/v.

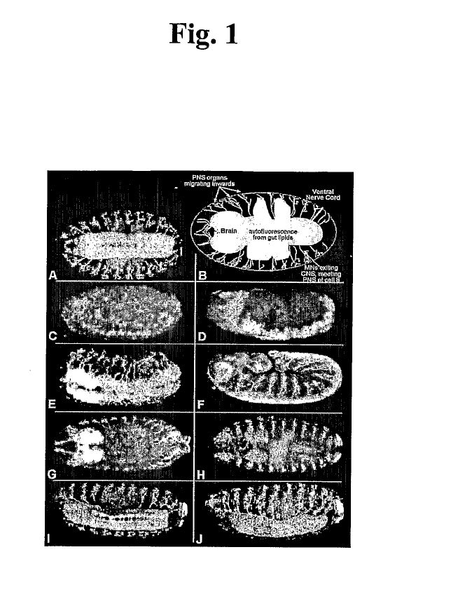

Gamma secretase is an enzyme complex that cleaves type I transmembrane proteins. Inhibitors of this complex inhibit the cleavage of gamma secretase substrates, including Notch, a protein essential for normal development at all embryonic stages. A dose response study using gamma secretase inhibitors on developing wild type embryos, generated correspondingly severe Notch phenotypes that were identical to phenotypes reported for Notch genetic hypomorphs. The sensitivity of these assays was extreme, with mild phenotypes detectable at even femtomolar concentrations (FIG. 1).

### 2. Example 2

### Screen Designed Using the Amyloidgenic Peptide Associated with Alzheimer's Disease

Transgenic Drosophila strains were made in which Abeta₁₋₄₀ and Abeta₁₋₄₂ were expressed throughout the CNS with pan-neuronal drivers. The uncleaved form of these peptides (known as CT100) were used and expressed with identical pan-neuronal drivers.

Alzheimer's Disease is characterized by dementia and deposition of amyloid containing plaques throughout the brain. No one type of neuronal sub-population is affected in this neurodegeneration. Furthermore, it was not known whether cells are insulted by an as yet unknown extracellular toxin, killed, and then dump their intracellular stores of undigestable amyloidgenic peptides into the extracellular space, where they accumulate as plaques, or whether the amyloidgenic peptides are themselves toxic intracellularly, accumulate to lethal levels and kill the neurons, leaving the Abeta plaques behind as the remnant ghosts of neurons that once contained them. In an effort to test both possibilities, the following transgenic strains of Drosophila melanogaster were made: UAS-Abeta₁₋₄₀, UAS-Abeta₁₋₄₂, and UAS-CT100.

Because these proteins accumulate within neurons, the constructs were duplicated and made with insect cuticle protein signal sequences (ss) as well, so that they would be secreted into the extracellular space. These strains then were: UAS-ₛₛAbeta₁₋₄₀, UAS-ₛₛAbeta₁₋₄₂, and UAS-ₛₛCT100.

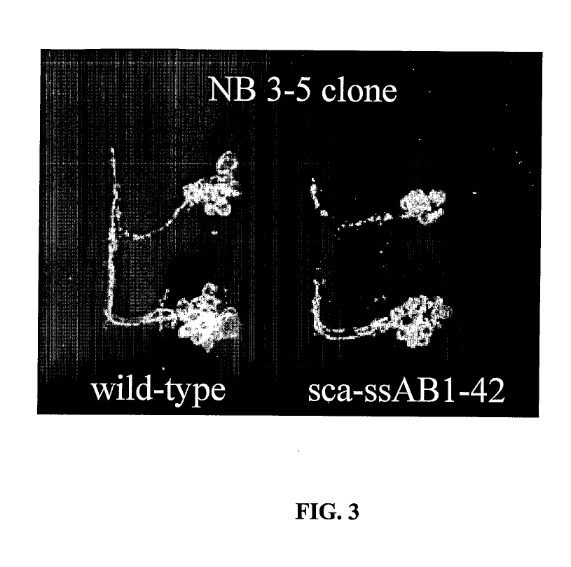

Taking advantage of the UAS-GAL4 system (Brand and Perrimon, 1993) for the amplification and specific misexpression of reporter genes, pan-neuronal drivers were used to misexpress these peptides in every cell of the embryonic CNS (FIG. 3).

A model has been proposed by which Alzheimer's Disease arises from impaired axonal transport of vesicles and organelles along microtubules. In this model, APP is packed into vesicles with \tilde{β} and \tilde{γ} secretase, the vesicles are transported via light chain I of kinesin along microtubules to the synapse, where the secretases function to cleave APP, and in the process, liberate vesicles from microtubules (Gunarwardena and Goldstein, 2001; Kamal et al, 2001).

When DiI labeling experiments were performed to examine the single cell defects associated with the misexpression of Abeta peptides pan-neuronally, it was observed that the population of neurons most strongly affected were longitudinal projection neurons. These neurons consistently failed to extend beyond segment boundaries in this gain of function background. When staining was done with fascicular antibodies, clear breaks could be visualized at segment boundaries that typically extended for 25% of the Anteroposterior length of the hemisegment.

### 3. Example 3

### High Throughput Screen of Chemical Compounds Using Drosophila Melanogaster Embryos

Screenable genetic phenotype in Drosophila melanogaster for human neurodegenerative diseases can be observed by the following method. Drosophila embryos are aliquoted into 96 well plates. Chemical compounds are then delivered to embryos in 96 well plates. An assay of phenotypic improvement is then carried out using seed fill algorithms.

a) Sorting of Embryos

Because heterozygous embryos appeared wild type, an embryo sorter from Union Biometrica was used to achieve two important goals: 1) heterozygous embryos (which had normal CNS phenotypes in comparison to homozygous sibs) were separated from homozygous embryos; 2) homozygous embryos can be precisely aliquoted into 96 well plates containing drugs in 1 μM concentrations, in 1% DMSO.

Because embryos are sorted using “Liquid Gold”, which might generate as much as 1-2 ul volume of liquid per embryo per well, higher 1% DMSO concentration was used, assuming the final concentration would be 50-60% DMSO, concentrations that were known to be as effective in the gamma secretase inhibitor experiment as the 1% DMSO concentrations.

b) Delivery of Compounds

It is necessary to demonstrate that compounds can be delivered into Drosophila embryos, long believed to be impossible, because of the presence of the vitelline membrane, a thick chitinous structure that appears to be impervious to most biologically active molecules, including antibodies.

First it was shown that DMSO was not deleterious to normal embryonic development. A dose-response curve was performed, and it was demonstrated that normal development occurs in 100% of embryos in concentrations of DMSO as high as 5%.

It was then demonstrated that small molecules could be delivered to embryos through an intact vitelline membrane. This was tested using two approaches. Fluorescent size markers available from Molecular Probes Corporation were used, and it was demonstrated that fluorescently tagged molecules could enter into embryos with the cut-off size being 3 Kd. Because of the high level of autofluorescence in Drosophila embryos, the experiment was repeated using commercially available inhibitors of gamma secretase and it was demonstrated that drugs could enter into embryos with great ease, using 1% DMSO in PBS as a vehicle. Also, defects could be detected with great sensitivity in these embryos, with known Notch phenotypes being detectable at femtomolar concentrations.

When a transgenic line is generated that has lethal insertion sites, it is necessary to maintain the parental lab stocks with fluorescently tagged balancer chromosomes. (It is important to note that the need for balancer chromosomes will vary from line to line, and is primarily a function of the disruption at the insertion site). The parental lines themselves were stained and the axonal disruptions observed from the Abeta misexpression background were never found.

c) The Read-Out Assay

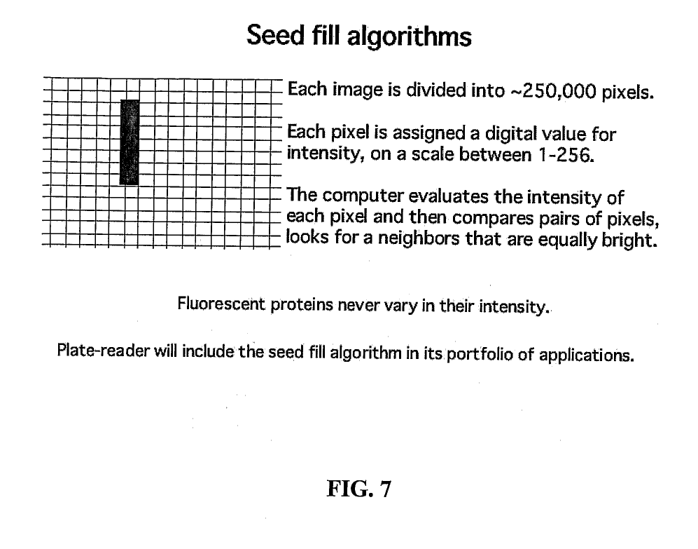

A seed-fill algorithm can be used as a rapid readout for this screen. Seed fill algorithms work as shown in FIG. 7. Each image is divided into approximately 250,000 pixels, and each pixel is assigned a digital value for intensity, on a scale between 1-256. The computer evaluates the intensity of each pixel and then compares pairs of pixels and looks for neighbors that are equally bright. Fluorescent proteins never vary in their intensity, and plate readers can be used with seed fill algorithms. Essentially, the algorithm evaluates contiguous lines of pixels. So if membrane tagged fluorescent axons (pixels) are only 100 pixels long in the untreated controls, then a drug that ameliorates the phenotype might be expected to improve this number by what would be a statistically significant amount (determined, of course, by the variation in the controls). The algorithm can be designed to fit the plate reader software.

### 4. Example 4

### Screening of Compounds for an Effect on Parkinson's Disease

The Parkinson's Disease model described above is used. A commercially available compound collection is it aliquoted into 96 well plates in 10 nM concentrations (Genesys Corporation, USDA). These collections are blindly seeded with positive and negative controls. Rotenone and other molecules known to be specific dopaminargic toxins serve as negative controls, and the commercially available agonist of Nurr1 (A receptor and transcription factor that directly results in dopaminergic neuron production) is a powerful positive control. The seed fill algorithm is modified to detect distance between dopaminergic neurons as a read-out rather than axonal length.

### 5. Example 5

### Determining DMSO Concentrations

Broad dilution series were made of DMSO in PBS. Approximately 50 eggs were placed in each well of a 96 well plate containing these solutions. They were placed at 18° C. or at 25° C. and hatched offspring were counted at the end of embryonic development (36 hours at 18° and 24 hours at 25°). If larvae failed to hatch, particular defects were noted, and percentages that hatched were noted in each dose range. 100% of eggs produced normal larvae at concentrations as high as 5% in PBS, pH 7.2. At 7.5% DMSO the percentage declined dramatically to approximately 50% and by 10% DMSO, the percentage hatching was only ˜10%. At 25°, the percentages surviving in solutions above 5% DMSO were always less than those kept at 18°.

### 6. Example 6

### Depression and the Wolframin Gene

Wolframin is a gene that is strongly linked to depression in humans. Homozygous patients exhibit diabetes, optic atrophy and deafness, severe intellectual impairment and mood disorders and death by age 30. Heterozygous individuals are phenotypically wildtype with the exception of a marked increase in depression; 26 times as many Wolframin heterozygotes seek treatment for depression as compared to wild type siblings. This gene encodes a 9 pass transmembrane protein, which appears to function in subcellular vesicular trafficking.

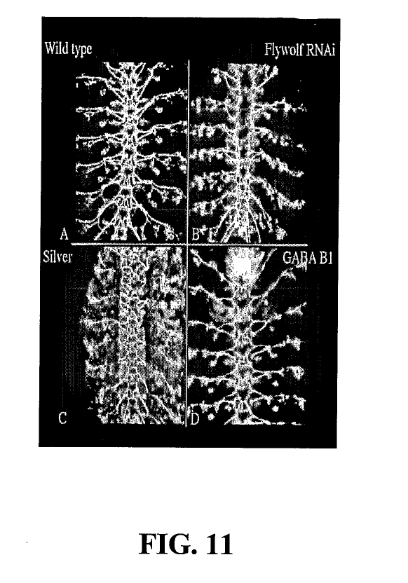

There is one Drosophila ortholog of this gene and it too encodes a 9 pass transmembrane protein albeit with relatively weak homology. There are no extant mutants and the region appears to be chromatin protected in Drosophila. An RNAi approach was used to characterize loss of function phenotypes (FIG. 11, panel B). Strong functional conservation has been validated between the orthologs, and a loss of function mutant can be generated by site-directed mutagenesis.

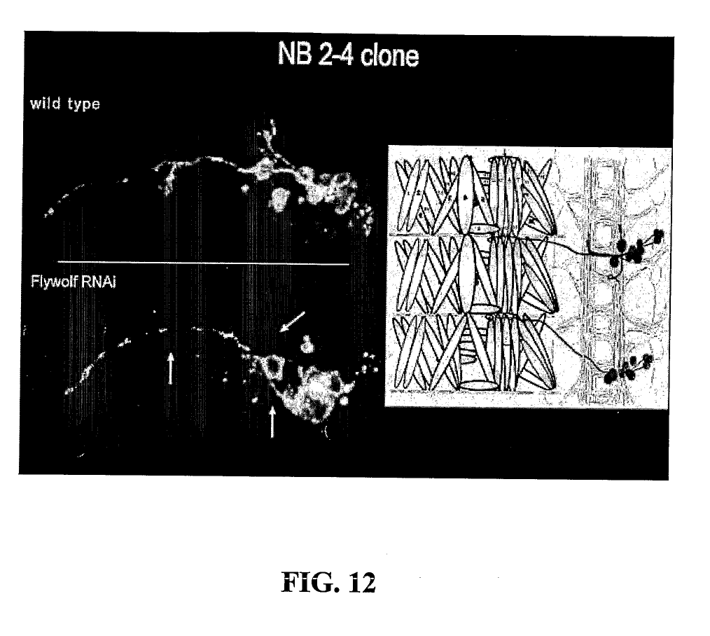

22C10 staining of Flywolf loss of function (RNAi) embryos reveals commissural defects that vary by anteroposterior position. Wild type embryos were injected with dsRNA for Flywolf and were allowed to develop to the end of embryonic development. They were then filleted, fixed and stained with a monoclonal antibody (22C10) that stains pioneer neurons of each of the major tracts of the embryonic ventral nerve cord. Images were collected on a Biorad Microradiance 2000 confocal microscope. Also, the RNAi and DiI labeling protocol was followed to generate the single cell clones (FIG. 12), in embryos in which flywolf function had been removed by RNAi. The NB 2-4 clone in loss of function Flywolf backgrounds generates contralaterally projecting motoneurons, but fails to project local interneuronal projections contralaterally. The loss of commissural interneuronal projection occurred in every clone examined.

These data are consistent with reports from vertebrate autopsies suggesting that the mood disorders associated with Wolfram's Syndrome are correlated with disruptions of the small cell layers of the cortex and show that the subcellular pathology appears to be similar in flies and humans. Experiments that demonstrate a strong functional conservation between fly and human version of the Wolframin protein have been conducted.

### 7. Example 7

### Neuronal Toxicity Assays

Using Molecular Probe's TUNEL kit, it was found that pan-neuronal misexpression of Abeta 1-42 generated wide-spread neuronal death in flies. Using an antibody specific for Abeta 1-42, it was found that embryonic ventral nerve cords can generate amyloid peptides, and cell death due to misexpression of Abeta does not occur only in the cells misexpressing Abeta, which is not unexpected as the peptides are secreted.

### E. REFERENCES

Throughout this application, various publications are referenced. The disclosures of these publications in their entireties are hereby incorporated by reference into this application in order to more fully describe the state of the art to which this invention pertains.
Campos-Ortega, J. A., and Hartenstein, V., eds., The Embryonic Development of Drosophila melanogaster, 2nd ed. (1996). Springer Verlag Press, Berlin.
King, R. C. (1970) Ovarian Development in Drosophila Melanogaster. Academic Press, New York.
Drew, J (1999) In Quest of Tomorrow's Medicines. springer Verlag, New York.
Mitchison T J, and J. Sedat (1983). “Localization of antigenic determinants in whole Drosophila embryos” Dev Biol 99(1):261-4
Christopher A. Lipinski, Franco Lombardo, Beryl W. Dominy, Paul J. Feeney “Experimental and computational approaches to estimate solubility and permeability in drug discovery and development settings”, Adv. Drug Delivery Rev., 1997, 23(1-3), 3-25.
Rubin, G. M. and A. C. Spradling. 1982. Genetic transformation of Drosophila with transposable element vectors. Science 218:348-353.
Salz, H. K., T. W. Cline and P. Schedl. 1987. Functional changes associated with structural alterations induced by mobilization of a P element inserted in the Sex-lethal gene of Drosophila. Genetics 117:221-231.
Searles, L. L., R. S. Jokerst, P. M. Bingham, R. A. Voelker and A. L. Greenleaf. 1982. Molecular cloning of sequences from a Drosophila RNA polymerase II locus by P element transposon tagging. Cell 31:585-592.
Sentry, J. W. and K. Kaiser. 1994. Application of inverse PCR to site-selected mutagenesis of Drosophila. Nucleic Acids Res 22:3429-30.
Serano, T. L., H. K. Cheung, L. H. Frank and R. S. Cohen. 1994. P element transformation vectors for studying Drosophila melanogaster oogenesis and early embryogenesis. Gene. 138:181-186.
Spradling, A. C. 1986. P element-mediated transformation. pp. 175-197 in Drosophila: A Practical Approach, edited by D. B. Roberts. IRL Press, Oxford.
The FlyBase Consortium (2003). The FlyBase database of the Drosophila genome projects and community literature. Nucleic Acids Research 31:172-175. http://flybase.org/
Spradling, A. C. and G. M. Rubin. 1982. Transposition of cloned P elements into Drosophila germ line chromosomes. Science 218:341-347.
Ashburner, M. 1989. Drosophila, A laboratory manual. Cold Spring Harbor Press, Cold Spring Harbor, N.Y.
Cary, L. C. et al. Transposon mutagenesis of baculoviruses: analysis of Trichoplusia ni transposon IFP2 insertions within the FP-locus of nuclear polyhedrosis viruses. Virology 172, 156-69 (1989).
Medhora, M. M., Maruyama, K. and Hartl, D. L. (1991) Genetics 128:311.
Hirschmann, et al., 1991. Angew. Chem. Int. Ed. Engl. 30:1278-1301
Schulz, et al., 1955. Cancer Res. 3(suppl.): 86-100; Schuler, et al., 1982. Terat. Carcin. Mutag. 2:293-301
Casso, D., Ramirez-Weber, F., Komberg, T. GFP-Tagged balancer chromosomes for Drosophila melanogaster. Mechanisms of Development, 91: 451-454, 2000.
Furlong, E., Profitt, D., Scott, M. Automated sorting of live transgenic embryos. Nature Biotechnology, 19: 153-156, 2001.
Lawrence, Peter. (1992) The Making of a Fly, The Genetics of Animal Design. Blackwell Science Ltd, Oxford.
Scangos, Nat. Biotechnol. (1997) 15:1220-1221
Lastowski-Perry et al, J. Biol. Chem., 260:1527, 1985
B. J. Bond et al, Mol. Cell. Biol., 6: 2080, 1986
Brand & Perrimon, Development (1993) 118: 401-415
Phelps & Brand, Methods (April 1998) 14:367-379
Steller & Pirrotta, Mol. Cell. Biol. 6:1640-1649, 1986
Christopher A. Lipinski, Franco Lombardo, Beryl W. Dominy, Paul J. Feeney, Adv. Drug Delivery Rev., 1997, 23(1-3), 3-25.
Roberts R. W. and Szostak J. W. Proc. Natl. Acad. Sci. USA, 94(23)12997-302 (1997).
Brand A H and N Perrimon (1993). “Targeted gene expression as a means of altering cell fates and generating dominant phenotypes.” Development 118(2): 401-15.
Brand A, et al (1994). “Ectopic expression in Drosophila”. Methods Cell Biol 44: 635-54.
Schmid A, et al, (1999). “Clonal analysis of Drosophila embryonic neuroblasts: neural cell types, axon projections and muscle targets.” Development 126: 4653-89.
Feany M B, and W M Bender (2000). “A Drosophila Model of Parkinson's Disease”. Nature 404: 394-8.
Pendleton R G, et al (2002). “Effects of Pharmacological Agents upon a Transgenic Model of Parkinson's Disease in Drosophila melanogaster.” The Journal of Pharmacology and Experimental Therapeutics 300(1): 91-6.
Auluck P K et al (2002). “Chaperone Suppression of α-Synuclein Toxicity in a Drosophila Model for Parkinson's Disease”. Science 295: 865-8
Drews J, (2000). “Drug Discovery: A historical Perspective”. Science 287: 1960-4.
Gunawardena S, and L S B Goldstein (2001). “Disruption of Axonal Transport and neuronal Viability by Amyloid Precursor Protein Mutations in Drosophila.” Neuron 32: 389-401
Kamal A, et al (2001). “Kinesin-mediated axonal transport of a membrane compartment containing B-secretase and presenilin-1 requires APP.” Nature 414: 643-8.

What is claimed is:

1. A method of manipulating an invertebrate animal embryo, comprising delivering a composition to the embryo.

2. A method of delivering a composition to an invertebrate animal embryo comprising:
a) dechorionating the embryo;
b) exposing the embryo to DMSO; and
c) delivering the composition to the embryo.

3. A method of making an invertebrate animal embryo useful for screening compounds comprising:
a) dechorionating the embryo; and
b) exposing the embryo to DMSO.

4. The method of claim 3, wherein the embryo has been genetically modified.

5. A method of screening a candidate compound for its effect on a disease comprising:
a) administering the compound to an invertebrate animal embryo; and
b) assaying the effect of the compound on the embryo.

6. A method of screening a candidate compound for its effect on a disease comprising:
a) administering the compound to an invertebrate animal embryo;
b) comparing phenotypes of the transgenic embryo treated with the compound to a transgenic invertebrate embryo not treated with the compound, wherein a difference in the phenotypes is indicative of an alleviating activity of the candidate compound.

7. A method of screening a compound for its effect on an invertebrate embryo comprising
a) dechorionating the embryo,
b) incubating the dechorionated embryo with the compound and DMSO, and
c) assaying the effect of the compound on the embryo.

8. A kit for use in screening compounds, said kit comprising transgenic invertebrate animal embryos and a suitable container.

9. A method of making a genetic assay for a neurodegenerative disease, comprising:
a) introducing a nucleic acid into an invertebrate animal, wherein the nucleic acid encodes a peptide associated with a phenotype of the neurodegenerative disease;
b) introducing a fluorescent protein into the invertebrate animal of step a;
c) introducing a UAS-Gal4 expression system into the invertebrate animal of step a;
d) expressing the phenotype in an embryo of the invertebrate animal of step c; and
e) assaying the phenotype.

10. The method of claim 9, wherein the peptide associated with a phenotype of the neurodegenerative disease is an amyloidgenic peptide.

11. The method of claim 10, wherein the amyloidgenic peptide is Abeta₁₋₄₂.

12. The method of claim 10, wherein the amyloidgenic peptide is Abeta₁₋₄₀.

13. The method of claim 9, wherein a signal sequence is also introduced into the invertebrate animal.

14. A method of screening a candidate compound for its effect on a disease comprising:
a) introducing a nucleic acid into an invertebrate animal, wherein the nucleic acid encodes a peptide associated with a phenotype of the disease,
b) expressing the phenotype in an embryo of the invertebrate animal of step a;
c) aliquoting the embryos of step c into wells of a plate;
d) delivering a test compound to the well of the plate of step d; and
e) screening for a change in the phenotype associated with the disease, a change indicating a compound with an effect upon the disease.

15. A method of making a genetic assay comprising:
a) introducing a nucleic acid into an invertebrate animal, wherein the nucleic acid encodes a peptide associated with a phenotype of a disease;
b) expressing the phenotype in an embryo of the invertebrate animal of step a;
c) aliquoting the embryos of step b into wells of a plate; and
d) delivering a test compound to the well of the plate of step c.

16. The method of any one of claims 5-15, wherein the disease is Parkinson's disease.

17. The method of any one of claims 5-15, wherein the disease is Alzheimer's disease.

18. The method of any one of claims 5-15, wherein the disease is depression.

19. A method of introducing a compound into an invertebrate embryo, comprising administering to the embryo an effective amount of DMSO.

20. The method of claim 19, wherein the effective amount of DMSO is 0.1% to 10%.

21. The method of claim 20, wherein the effective amount of DMSO is 1% to 5%.

22. A method of screening for a test compound with an effect on a phenotype associated with a neurodegenerative disease comprising:
a) placing transgenic embryos in a read out assay;
b) delivering a test compound to the read out assay of step a;
c) scoring the embryos for the ability to extend axons across segment boundaries, said ability indicative of a test compound with an effect on a phenotype associated with a neurodegenerative disease.

23. The method of claim 0, wherein transgenic embryos express a membrane-tagged fluorescent protein.

24. A method of scoring a plate comprising:
a) placing transgenic embryos expressing a phenotype associated with a disease in a read out assay;
b) delivering a compound to the read out assay; and
c) using a seed fill algorithm to score the embryos, wherein the algorithm evaluates contiguous lines of pixels, wherein a compound that ameliorates the phenotype gives a higher score in comparison to a control embryo.


SEQUENCE LISTING (27 sequences)

SEQ ID NO: 1 - DNA, 2479 bp, Artificial Sequence
  Description of Artificial Sequence; note = synthetic construct
ccccctcggccccgcgcgtcgcctgtcctccgagccagtcgctgacagccgcggcgccgcgagcttctcctctcctcacgaccgagagcagtcattatggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatgaggaaggtcttcctgttttcaccatctttctaatctttttccagcttgagggaggcggtatccacctgcagcccttttagtggtggtgtctcactctttcttctctctttgtcccggataggctaatcaatacccttggcactgatgggcactggaaaacatagagtagacctgagatgctggtcaagccccctttgattgagttcatcatgagccgttgctaatgccaggccagtaaaagtataacagcaaataaccattggttaatctggacttatttttggacttagtgcaacaggttgaggctaaaacaaatctcagaacagtctgaaatacctttgcctggatacctctggctccttcagcagctagagctcagtatactaatgccctatcttagtagagatttcatagctatttagagatattttccattttaagaaaacccgacaacatttctgccaggtttgttaggaggccacatgatacttattcaaaaaaatcctagagattcttagctcttgggatgcaggctcagcccgctggagcatgagctctgtgtgtaccgagaactggggtgatgttttacttttcacagtatgggctacacagcagctgttcaacaagagtaaatattgtcacaacactgaacctctggctagaggacatattcacagtgaacataactgtaacatatatgaaaggcttctgggacttgaaatcaaatgtttgggaatggtgcccttggaggcaacctcccattttagatgtttaaaggaccctatatgtggcattcctttctttaaactataggtaattaaggcagctgaaaagtaaattgccttctagacactgaaggcaaatctcctttgtccatttacctggaaaccagaatgattttgacatacaggagagctgcagttgtgaaagcaccatcatcatagaggatgatgtaattaaaaaatggtcagtgtgcaaagaaaagaactgcttgcatttctttatttctgtctcataattgtcaaaaaccagaattaggtcaagttcatagtttctgtaattggcttttgaatcaaagaatagggagacaatctaaaaaatatcttaggttggagatgacagaaatatgattgatttgaagtggaaaaagaaattctgttaatgttaattaaagtaaaattattccctgaattgtttgatattgtcacctagcagatatgtattacttttctgcaatgttattattggcttgcactttgtgagtattctatgtaaaaatatatatgtatataaaatatatattgcataggacagacttaggagttttgtttagagcagttaacatctgaagtgtctaatgcattaacttttgtaaggtactgaatacttaatatgtgggaaacccttttgcgtggtccttaggcttacaatgtgcactgaatcgtttcatgtaagaatccaaagtggacaccattaacaggtctttgaaatatgcatgtactttatattttctatatttgtaactttgcatgttcttgttttgttatataaaaaaattgtaaatgtttaatatctgactgaaattaaacgagcgaagatgagcaccaaaaaaaaaaaaaaaa

SEQ ID NO: 2 - DNA, 722 bp, Artificial Sequence
  Description of Artificial Sequence; note = synthetic construct
aaggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaaaaaaaaaaaaaaaaaaa

SEQ ID NO: 3 - protein, 36 aa, Artificial Sequence
  Description of Artificial Sequence; note = synthetic construct
DAEFRHDSGYEVHHQKLVFFAEDVGSNKGAIIGLMV

SEQ ID NO: 4 - protein, 38 aa, Artificial Sequence
  Description of Artificial Sequence; note = synthetic construct
DAEFRHDSGYEVHHQKLVFFAEDVGSNKGAIIGLMVIA

SEQ ID NO: 5 - protein, 54 aa, Artificial Sequence
  Description of Artificial Sequence; note = synthetic construct
DAEFRHDSGYEVHHQKLVFFAEDVGSNKGAIIGLMVIATVIVITLVMLKKQYTS

SEQ ID NO: 6 - protein, 95 aa, Artificial Sequence
  Description of Artificial Sequence; note = synthetic construct
DAEFRHDSGYEVHHQKLVFFAEDVGSNKGAIIGLMVIATVIVITLVMLKKQYTSIHHGVVEVDAAVTPEERHLSKMQQNGYENPTYKFFEQMGNC

SEQ ID NO: 7 - protein, 253 aa, Artificial Sequence
  Description of Artificial Sequence; note = synthetic construct
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 8 - protein, 695 aa, Artificial Sequence
  Description of Artificial Sequence; note = synthetic construct
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 9 - protein, 360 aa, Artificial Sequence
  Description of Artificial Sequence; note = synthetic construct
MEERERGARSAGAGSPARPPSPRLDVSSDSFDPLLALYAPRLPPIPYPNAPCFNNVAEYESFLRTGVRGGGRGRGRARGAAAGSGVPAAPGPSGRTRRRPDAPAPDPERIQRLRRLMVAKEEGDGAAGAGRRGPGRSRKAPRNVLTRMPLHEGSPLGELHRCIREGVKVNVHIRTFKGLRGVCTGFLVAFDKFWNMALTDVDETYRKPVLGKAYERDSSLTLTRLFDRLKLQDSSKKEADSKSAVEDSTLSRYSQTSTWKLASVWGRADTGRGSHKRSRSVPSSLQASAREESRSELSGRTTRTDGSSVGGTFSRATTLSRGQSRKKKRKPKVDYQQVFTRHINQIFIRGENVLLVHLAQ

SEQ ID NO: 10 - protein, 226 aa, Artificial Sequence
  Description of Artificial Sequence; note = synthetic construct
MSDETNAAVWDDSLLVKTYDESVGLAREALARRLADSTNKREEENAAAAEEEAGEISATGGATSPEPVSFKVGDYARATYVDGVDYEGAVVSINEEKGTCVLRYLGYENEQEVLLVDLLPSWGKRVRREQFLIAKKDEDEQLSRPKASAGSHSKTPKSSRRSRISGGLVMPPMPPVPPMIVGQGDGAEQDFVAMLTAWYMSGYYTGLYQGKKEASTTSGKKKTPKK

SEQ ID NO: 11 - protein, 282 aa, Artificial Sequence
  Description of Artificial Sequence; note = synthetic construct
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMEMLA

SEQ ID NO: 12 - protein, 262 aa, Artificial Sequence
  Description of Artificial Sequence; note = synthetic construct
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKIIPPPPPICPDSLDDADALGSMLISWYMSGYHTGYYMGFRQNQKEGRCSHSLN

SEQ ID NO: 13 - protein, 250 aa, Artificial Sequence
  Description of Artificial Sequence; note = synthetic construct
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKIIPPPPPICPDSLDDADALGSMLISWYMSGYHTGYYMEMLA

SEQ ID NO: 14 - protein, 294 aa, Artificial Sequence
  Description of Artificial Sequence; note = synthetic construct
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 15 - protein, 140 aa, Artificial Sequence
  Description of Artificial Sequence; note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 16 - protein, 3139 aa, Artificial Sequence
  Description of Artificial Sequence; note = synthetic construct
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVKQLKVMSQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 17 - DNA, 512 bp, Artificial Sequence
  Description of Artificial Sequence; note = synthetic construct
agcaatgttttcctgtgttgtttatgcttaaaaagcgattttaggcgttattaaggacgcgaaaatggacaaatccaggtccagtgcgtacgacaagttcgttggcttcgtggaacaactgcgaaatacggaatgcagccaaaagcagaagatcacctgctttcagcagattgccgagtgcatcatgtcgccctcgctggcgggccacatcaactatgccgcccactgcggcacggccacgaatgttctgctgctgttctgcgaggatgtggattccgtggttcggatgagcgccgaggaaaacctaaataaaatactgaggtcactcgagaaaacccgggtcagtcgcattcttatggatctgtatggtgaaattaagcgtaatggtaaccagagatccctgaggatttgcctaaaccttttctcctactatgctccacaaataaaggagaaacacatcaagtggtatgccgtgagattgctccaatgtatgaccaccatttcccagcgca

SEQ ID NO: 18 - protein, 154 aa, Artificial Sequence [sequence omitted]

SEQ ID NO: 19 - DNA, 714 bp, Artificial Sequence
  Description of Artificial Sequence; note = synthetic construct
ctacggtcacaccatagaagatacctggaaagttctcaacttttttcgttttgataaattgattaattcattcgaaatggtggttaaagctgtctgcgtaattaacggcgatgccaagggcacggttttcttcgaacaggagagcagcggtacgcccgtgaaggtctccggtgaggtgtgcggcctggccaagggtctgcacggattccacgtgcacgagttcggtgacaacaccaatggctgcatgtcgtccggaccgcacttcaatccgtatggcaaggagcatggcgctcccgtcgacgagaatcgtcacctgggcgatctgggcaacattgaggccaccggcgactgccccaccaaggtcaacatcaccgactccaagattacgctcttcggcgccgacagcatcatcggacgcaccgttgtcgtgcacgccgatgccgatgatcttggccagggtggacacgagctgagcaagtcaacgggcaacgctggtgcccgcatcgggtgcggcgttattggcattgccaaggtctaagcgataatctattccgatgtcggccactgtgctgatctactctatttagcactacccactggagatatacaaacgatatacatacttctaaacataaatacatagcctgtggtctgttagttgatacgcaacctttgaggttcaataaattggtgttttgaaattgccccataaac

SEQ ID NO: 20 - protein, 352 aa, Artificial Sequence
  Description of Artificial Sequence; note = synthetic construct
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 21 - protein, 360 aa, Artificial Sequence
  Description of Artificial Sequence; note = synthetic construct
MADVLEKSSLLDAVPPLGDPHPPLPHQQLQQEAAAAAAANAAPPAPPQQQQPPPHQLQQQQPQQQQLQQKPANARANQDQKEGDNDSGVDESTQEKDRNGPNSPSSPVKTPTSTSSKPDKSGTSRPPSATPSNKSAPKSRSASKNRLLLKTPEPEPVKKVPMNKVQVGHAPSPNLKAVRSKIGSLDNATYKPGGGHVKIESKKIDIKAAPRIEAKNDKYMPKGGEKKIVTTKLQWNAKSKIGSLENAAHKPGGGDKKIETLKMDFKDKAKPKVGSTANVKHQPGGGDIKIQTQKLEIKAQSKVGSLDNVKHKPGGGEKKIFDDKDYLKNVEHSVALTTPPTQSPLPSMTASGADENLNQQ

SEQ ID NO: 22 - protein, 585 aa, Artificial Sequence
  Description of Artificial Sequence; note = synthetic construct
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 23 - protein, 721 aa, Artificial Sequence
  Description of Artificial Sequence; note = synthetic construct
MTITYTGEVATCRGFGCFLKLLLRWRGSIYKLVWLDLLAFLTIYYAINMVYRFGLNPAQKETFEAIVQYCDSYRELIPLSFVLGFYVSIVMTRWWNQYTSIPWPDPIAVFVSSNVHGQDERGRMMRRTIMRYVCLCLTMVLANVSPRVKKRFPGLNNLVEAGLLNDNEKTIIETMNKAFPRPSKHWLPIVWAASIITRARKEGRIRDDFAVKTIIDELNKFRGQCGLLISYDTISVPLVYTQVVTLAVYSYFLTCCMGQQWTDGKVVGNTTYLNKVDLYFPVFTTLQFFFYMGWLKVAESLINPFGEDDDDFEVNWMVDRNLQVSYLIVDEMHHDHPELLKDQYWDEVFPNELPYTIAAERFRENHPEPSTAKIEVPKNAAMPSTMSSVRIDEMADDASGIHFSAGNGKMRLDSSPSLVSVSGTLSRVNTVASALKRFLSRDDSRPGSATPSQDQPYKFPASASSASLSGAVVGSATSAGKPAGSLRITQQVIEEVDEQATITSMRANDPRPNVMDIFAQTSSGAGTSGPLQPPPAHSEPVDIPSRPPSYNRAQSQYEPNLFPPGGVDALLSTSAPAGGSPLLLSNAATAPSSPVGESSKSLYDPQKGASRETVESMDLRSSTDLLGDAAVQPEDEGDDFDKLKAEREKEKLMRQQKNLARTISTAPGMEATAVPMVPMVPVNVAVQQAQLQPVASSADLLAGGDQFSNSTMKSEDAINGS

SEQ ID NO: 24 - protein, 890 aa, Artificial Sequence
  Description of Artificial Sequence; note = synthetic construct
MDSNTAPLGPSCPQPPPAPQPQARSRLNATASLEQERSERPRAPGPQAGPGPGVRDAAAPAEPQAQHTRSRERADGTGPTKGDMEIPFEEVLERAKAGDPKAQTEVGKHYLQLAGDTDEELNSCTAVDWLVLAAKQGRREAVKLLRRCLADRRGITSENEREVRQLSSETDLERAVRKAALVMYWKLNPKKKKQVAVAELLENVGQVNEHDGGAQPGPVPKSLQKQRRMLERLVSSESKNYIALDDFVEITKKYAKGVIPSSLFLQDDEDDDELAGKSPEDLPLRLKVVKYPLHAIMEIKEYLIDMASRAGMHWLSTIIPTHHINALIFFFIVSNLTIDFFAFFIPLVIFYLSFISMVICTLKVFQDSKAWENFRTLTDLLLRFEPNLDVEQAEVNFGWNHLEPYAHFLLSVFFVIFSFPIASKDCIPCSELAVITGFFTVTSYLSLSTHAEPYTRRALATEVTAGLLSLLPSMPLNWPYLKVLGQTFITVPVGHLVVLNVSVPCLLYVYLLYLFFRMAQLRNFKGTYCYLVPYLVCFMWCELSVVILLESTGLGLLRASIGYFLFLFALPILVAGLALVGVLQFARWFTSLELTKIAVTVAVCSVPLLLRWWTKASFSVVGMVKSLTRSSMVKLILVWLTAIVLFCWFYVYRSEGMKVYNSTLTWQQYGALCGPRAWKETNMARTQILCSHLEGHRVTWTGRFKYVRVTDIDNSAESAINMLPFFIGDWMRCLYGEAYPACSPGNTSTAEEELCRLKLLAKHPCHIKKFDRYKFEITVGMPFSSGADGSRSREEDDVTKDIVLRASSEFKSVLLSLRQGSLIEFSTILEGRLGSKWPVFELKAISCLNCMAQLSPTRRHVKIEHDWRSTVHGAVKFAFDFFFFPFLSAA

SEQ ID NO: 25 - protein, 813 aa, Artificial Sequence
  Description of Artificial Sequence; note = synthetic construct
MQYDLAKELLDNSIVEPNLAKGNQSQKAVNWLVSAAHNGHEDAVKLLRQCYNDGSGITAENTDEVRRCLAMTPGERAARKAARELFACLSNGNEHITPKQLERKMRRIYNLQRKRRRRDDDRSSSSSEGEQEPECEPLEDVPTIDLANVERRRLITEAHLVSAASNYSAGQMPSVNDALTLSVPDPRSLDHVPCFYRMIFHPLIFFTLFYHRLLNLIVSIPNVIPLSVRCSVLVAISWWSSRHMLPLVSYYLSLGIMIWATCKMLKTKQQFVDFRIWSGLFLSYGDQNIEADIAEHRFLRNNMKPYLYFFCAFICNLIVYPLVTDAWLPHSELTIISGALTFITMCVSMYASSHQLPDWLVIVSFAVNVLAKYPYEMDEVVSTRWRFLDLRVPTFSSFVIGNGIEFCLNCRTALYLFIPVLLIMMAKRSRWHGVYTFLIPHCVTLSWLQVCIATSQSSTVFGVMRAALGLAGIVLFLPLFGIVALLVPVFVAIDSLGLASEQLRWGSTALACGLVVVLSCILALNRATQKYITMLQLITAITTACLLVLPYMTSSFKDTPRFNAMPRAGLHSLSETNTLPWDRFHALCAQPVHEQPNKIKAQLRCSLLNGMPVIWEGSVTKVEISRVSNFLEDTIANYLPVWLGRMLRCLHGENISQHFKCDPKLDAQCEEWRSVFKTFNAQSGSCTLQRWNRYEYELLVKVGTKRSGRLLGRSTTTDVILRAHHDFGNFTRLLSEGDVVLFYGILHNSRLLADNVQVKLKTIECVECRSRDLGTASIERVVAASPMDARLQDLMRGIKYLLNALLNPLITFK

SEQ ID NO: 26 - protein, 351 aa, Artificial Sequence
  Description of Artificial Sequence; note = synthetic construct
MLETLRERLLSVQQDFTSGLKTLSDKSREAKVKSKPRTVPFLPKYSAGLELLSRYEDTWAALHRRAKDCASAGELVDSEVVMLSAHWEKKKTSLVELQEQLQQLPALIADLESMTANLTHLEASFEEVENNLLHLEDLCGQCELERCKHMQSQQLENYKKNKRKELETFKAELDAEHAQKVLEMEHTQQMKLKERQKFFEEAFQQDMEQYLSTGYLQIAERREPIGSMSSMEVNVDMLEQMDLMDISDQEALDVFLNSGGEENTVLSPALGPESSTCQNEITLQVPNPSELRAKPPSSSSTCTDSATRDISEGGESPVVQSDEEEVQVDTALATSHTDREATPDGGEDSDS

SEQ ID NO: 27 - protein, 288 aa, Artificial Sequence
  Description of Artificial Sequence; note = synthetic construct
MFGNLKKKLSSAIQEGLVISENLQQQYRQRVSSGNSGSSQASGITTPISPLGLNESLSSSRSSSLSLSAPFQLTDGVPSHLNVAAGCSLLAKYEDDWQQIHGANEKNAEKAAQIANQISGIQDQASHQHRIMSELNSSLAGIPTLIAQLQNSSQVLNSLEEMGKQLEIELEKLEDLREECELQEFILEQQFQLSRHKQKKLNELEQYRQQIAQKHQSKIKDQEQTLLKLQRERQAVFDDAFREDMEEYKQRGQLTKIQTTSNKLALEEVVLEANEVETKDALEQFLNG